SEQUENCE LISTING (62 sequences)

SEQ ID NO: 1 - protein, 16 aa, Artificial Sequence
  1A1 mAb VL CDR1
RSSQSLVYSDGNTYLN

SEQ ID NO: 2 - protein, 7 aa, Artificial Sequence
  1A1 mAb VL CDR2
KVSNRDS

SEQ ID NO: 3 - protein, 9 aa, Artificial Sequence
  1A1 mAb VL CDR3
MQSTHWPLT

SEQ ID NO: 4 - protein, 112 aa, Artificial Sequence
  1A1 mAb VL
EIVLTQSPLSLPVTLGQPASISCRSSQSLVYSDGNTYLNWFHQRPGQSPRRLIYKVSNRDSGVPERFSGSGSGTDFTLKISRVEAEDVGVYYCMQSTHWPLTFGGGTKVEIK

SEQ ID NO: 5 - protein, 219 aa, Artificial Sequence
  1A1 mAb IgG1 Light chain
EIVLTQSPLSLPVTLGQPASISCRSSQSLVYSDGNTYLNWFHQRPGQSPRRLIYKVSNRDSGVPERFSGSGSGTDFTLKISRVEAEDVGVYYCMQSTHWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  1A1 mAb VH CDR1
SYGIS

SEQ ID NO: 7 - protein, 17 aa, Artificial Sequence
  1A1 mAb VH CDR2
WISAYNGNTNYAQKLQG

SEQ ID NO: 8 - protein, 20 aa, Artificial Sequence
  1A1 mAb VH CDR3
AGTPTQILRYFDWLSQPFDY

SEQ ID NO: 9 - protein, 129 aa, Artificial Sequence
  1A1 mAb VH
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAGTPTQILRYFDWLSQPFDYWGQGTLVTVSS

SEQ ID NO: 10 - protein, 459 aa, Artificial Sequence
  1A1 mAb IgG1 Heavy chain
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAGTPTQILRYFDWLSQPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 14 aa, Artificial Sequence
  1A1 HBiTE CD3 VL CDR1
RSSTGAVTTSNYAN

SEQ ID NO: 12 - protein, 7 aa, Artificial Sequence
  1A1 HBiTE CD3 VL CDR2
GANKRAP

SEQ ID NO: 13 - protein, 9 aa, Artificial Sequence
  1A1 HBiTE CD3 VL CDR3
ALWYSNLWV

SEQ ID NO: 14 - protein, 110 aa, Artificial Sequence
  1A1 HBiTE CD3 VL
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIK

SEQ ID NO: 15 - protein, 565 aa, Artificial Sequence
  1A1 HBiTE (GPC3 X CD3) Light chain (GPC3 VL-linker-CD3 VLCL-mFc7.2)
EIVLTQSPLSLPVTLGQPASISCRSSQSLVYSDGNTYLNWFHQRPGQSPRRLIYKVSNRDSGVPERFSGSGSGTDFTLKISRVEAEDVGVYYCMQSTHWPLTFGGGTKVEIKGGGGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 16 - protein, 7 aa, Artificial Sequence
  1A1 HBiTE CD3 VH CDR1
GFTFNTY

SEQ ID NO: 17 - protein, 8 aa, Artificial Sequence
  1A1 HBiTE CD3 VH CDR2
RSKYNNYA

SEQ ID NO: 18 - protein, 14 aa, Artificial Sequence
  1A1 HBiTE CD3 VH CDR3
HGNFGSSYVSYFAY

SEQ ID NO: 19 - protein, 125 aa, Artificial Sequence
  1A1 HBiTE CD3 VH
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSS

SEQ ID NO: 20 - protein, 593 aa, Artificial Sequence
  1A1 HBiTE Heavy chain (GPC3 VH-linker-CD3 VHCH1-mFc7.2)
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAGTPTQILRYFDWLSQPFDYWGQGTLVTVSSGGGSSGGGGSGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 21 - protein, 15 aa, Artificial Sequence
  1A1 HBiTE light chain linker
GGGGSGGGGSGGGGS

SEQ ID NO: 22 - protein, 15 aa, Artificial Sequence
  1A1 HBiTE Heavy chain linker
GGGSSGGGGSGGGGS

SEQ ID NO: 23 - protein, 11 aa, Artificial Sequence
  64A mAb VL CDR1
RASQSISSYLN

SEQ ID NO: 24 - protein, 7 aa, Artificial Sequence
  64A mAb VL CDR2
AASSLQS

SEQ ID NO: 25 - protein, 9 aa, Artificial Sequence
  64A mAb VL CDR3
QQSYSTPLT

SEQ ID NO: 26 - protein, 107 aa, Artificial Sequence
  64A mAb VL
DVVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 27 - protein, 214 aa, Artificial Sequence
  6A4 mAb IgG1 Light chain
DVVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 113 aa, Artificial Sequence
  64A mAb VH
EVQLVQSGAEVKKPGASVKVSCKASGYTFTSYAMHWVRQAPGQRLEWMGWINAGNGNTKYSQKFQGRVTITRDTSTSTAYMELRSLRSDDTAVYYCARDPSHWGQGTLVTVSS

SEQ ID NO: 29 - protein, 443 aa, Artificial Sequence
  6A4 mAb IgG1 Heavy chain
EVQLVQSGAEVKKPGASVKVSCKASGYTFTSYAMHWVRQAPGQRLEWMGWINAGNGNTKYSQKFQGRVTITRDTSTSTAYMELRSLRSDDTAVYYCARDPSHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 557 aa, Artificial Sequence
  6A4 HBiTE (GPC3 X CD3) Light chain (GPC3 VL-linker-CD3 VLCL-mFc7.2)
DVVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 31 - protein, 577 aa, Artificial Sequence
  6A4 HBiTE (GPC3 X CD3) Heavy chain (GPC3 VH-linker-CD3 VHCH1-mFc7.2)
EVQLVQSGAEVKKPGASVKVSCKASGYTFTSYAMHWVRQAPGQRLEWMGWINAGNGNTKYSQKFQGRVTITRDTSTSTAYMELRSLRSDDTAVYYCARDPSHWGQGTLVTVSSGGGSSGGGGSGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 32 - protein, 12 aa, Artificial Sequence
  6A4 HBiTE light chain linker
GSGGGGSGGGGS

SEQ ID NO: 33 - DNA, 51 bp, Artificial Sequence
  primer
gaataagcttgccgccaccatggaatggagctgggtctttctcttcttcct

SEQ ID NO: 34 - DNA, 56 bp, Artificial Sequence
  primer
gtactctagattatttacccggagacagggagaggctcttctgcgtgtagtggttg

SEQ ID NO: 35 - DNA, 47 bp, Artificial Sequence
  primer
agtccgcggccgcgccaccatgggtgtgcccactcaggtcctggggt

SEQ ID NO: 36 - DNA, 38 bp, Artificial Sequence
  primer
gcatctcgagttaacactctcccctgttgaagctcttt

SEQ ID NO: 37 - DNA, 39 bp, Artificial Sequence
  primer
tgtgtgagttttgtcacaagatttgggctcaactttctt

SEQ ID NO: 38 - DNA, 36 bp, Artificial Sequence
  primer
tgtgacaaaactcacacatgtccaccgtgcccagca

SEQ ID NO: 39 - DNA, 59 bp, Artificial Sequence
  primer
cagcactgctctgttgcctggtcctcctgactggggtgagggccgaagtgcagctggtg

SEQ ID NO: 40 - DNA, 45 bp, Artificial Sequence
  primer
ggcatgtgtgagttttgtcacaagatttgggctcaactttcttgt

SEQ ID NO: 41 - DNA, 21 bp, Artificial Sequence
  primer
gtgacaaaactcacacatgcc

SEQ ID NO: 42 - DNA, 38 bp, Artificial Sequence
  primer
cgattctagaatcatttacccggggacagggagaggct

SEQ ID NO: 43 - DNA, 57 bp, Artificial Sequence
  primer
gcactgctctgttgcctggtcctcctgactggggtgagggccgatgttgtgatgact

SEQ ID NO: 44 - DNA, 38 bp, Artificial Sequence
  primer
cgattctagaatcaacactctcccctgttgaagctctt

SEQ ID NO: 45 - DNA, 68 bp, Artificial Sequence
  primer
gaatgcggccgcaaactacaagacagacttgcaaaagaaggcatgcacagctcagcactgctctgttg

SEQ ID NO: 46 - DNA, 41 bp, Artificial Sequence
  primer
gtgtaagcttaccatgggtgtgcccactcaggtcctggggt

SEQ ID NO: 47 - DNA, 32 bp, Artificial Sequence
  primer
caggtgtccactccgaaattgtgctgactcag

SEQ ID NO: 48 - DNA, 43 bp, Artificial Sequence
  primer
agggggatcctttgatctccaccttggtccctccgccgaaagt

SEQ ID NO: 49 - DNA, 41 bp, Artificial Sequence
  primer
gtgttctagagccgccaccatggaatggagctgggtctttc

SEQ ID NO: 50 - DNA, 38 bp, Artificial Sequence
  primer
ggcttacagatgccagatgtgaggtgcagctggtgcag

SEQ ID NO: 51 - DNA, 31 bp, Artificial Sequence
  primer
gatagagctcgaggagacggtgaccagggtt

SEQ ID NO: 52 - DNA, 64 bp, Artificial Sequence
  primer
tcagcactgctctgttgcctggtcctcctgactggggtgagggccgatgttgtgatgactcagt

SEQ ID NO: 53 - DNA, 46 bp, Artificial Sequence
  primer
gccagagccacctccgccggatcctttgatctccaccttggtccct

SEQ ID NO: 54 - DNA, 62 bp, Artificial Sequence
  primer
gcggccgcaaactacaagacagacttgcaaaagaaggcatgcacagctcagcactgctctgt

SEQ ID NO: 55 - DNA, 24 bp, Artificial Sequence
  primer
ggatccggcggaggtggctctggc

SEQ ID NO: 56 - DNA, 39 bp, Artificial Sequence
  primer
tgatctagaattatttacccggagacagggagaggctct

SEQ ID NO: 57 - DNA, 64 bp, Artificial Sequence
  primer
gctcagcactgctctgttgcctggtcctcctgactggggtgagggccgaagtgcagctggtgca

SEQ ID NO: 58 - DNA, 45 bp, Artificial Sequence
  primer
acctccgcctgagctccctccacctgaggagacggtgaccagggt

SEQ ID NO: 59 - DNA, 24 bp, Artificial Sequence
  primer
ggtggagggagctcaggcggaggt

SEQ ID NO: 60 - protein, 5 aa, Artificial Sequence
  64A mAb VH CDR1
SYAMH

SEQ ID NO: 61 - protein, 17 aa, Artificial Sequence
  64A mAb VH CDR2
WINAGNGNTKYSQKFQG

SEQ ID NO: 62 - protein, 4 aa, Artificial Sequence
  64A mAb VH CDR3
DPSH

(19) United States
(12) Patent Application Publication
(10) Pub. No.: US 2024/0368303 A1
(43) Pub. Date: Nov. 7, 2024

(54) ANTIBODIES AGAINST GPC3 AND USES THEREOF

(71) Applicant: ZHEJIANG SHIMAI PHARMACEUTICAL CO., LTD., Zhejiang (CN)

(72) Inventors: Zuoxiang Xiao, Hangzhou (CN); Jiaping Peng, Hangzhou (CN); Dongwen Zhou, Hangzhou (CN); Wei Zhou, Hangzhou (CN); Hangbin Miao, Hangzhou (CN); Guannv Wang, Hangzhou (CN)

(73) Assignee: ZHEJIANG SHIMAI PHARMACEUTICAL CO., LTD., Zhejiang (CN)

(21) Appl. No.: 18/691,036

(22) PCT Filed: Dec. 23, 2021
(86) PCT No.: PCT/CN2021/140644

§ 371 Date: Mar. 11, 2024

(30) Foreign Application Priority Data
Sep. 24, 2021 (WO) PCT/CN2021/120228

(51) Int. Cl. C07K 16/30; A61K 39/00; A61P 35/00

(52) U.S. Cl. CPC C07K 16/303; A61P 35/00; A61K 2039/505; C07K 2317/24; C07K 2317/31; C07K 2317/565; C07K 2317/73

(57) ABSTRACT

Disclosed herein are antibodies against GPC3 and uses thereof, specifically monoclonal antibodies against GPC3, bispecific antibodies against GPC3 and CD3, nucleic acids comprising the antibodies, vectors comprising the nucleic acids, and host cell comprising the nucleic acids or the vectors. Also disclosed are pharmaceutical compositions and conjugates comprising the antibodies, and therapeutic methods for using the antibodies.

### FIELD OF THE INVENTION

The present invention is directed to antibodies against GPC3, and uses of such antibodies, in particular their use in the treatment of cancers.

### BACKGROUND OF THE INVENTION

Glypicans (GPCs) represent a highly conserved family of heparan sulfate proteoglycans, which are attached to the plasma membrane via a C-terminal glycosyl-phosphatidylinositol (GPI) anchor. Six members of the GPC family so far have been identified in mammals: GPC1 to GPC6. Glypicans have similar structures including a 60-70 kDa core protein, which is linked to cell membrane via a GPI, and C-terminal heparan sulfate side chains. All GPC proteins are highly expressed during embryonic development and changes greatly in adults. In adults, GPC2 is no longer expressed; GPC3 is expressed in the ovary only; GPC5 is specifically expressed in the brain; and GPC1, GPC4 and GPC6 are widely expressed in various tissues.

It has been shown that GPCs are highly correlated with tumor development. GPC1 is associated with pancreatic cancer growth, migration and angiogenesis. GPC1 is also up-regulated in breast cancer, esophageal squamous cell carcinoma, and glioma, which suggests a poor prognosis. GPC2 is mainly associated with nerve system tumors, such as neuroblastoma. Some studies showed that GPC4 is highly expressed in pancreatic cancer, and GPC6 is up-regulated in ovarian cancer and positively correlated with prognosis. Of all the GPC family members, GPC3 is of the most interest and has been well-studied, which has been shown to be a potential marker for tumor diagnosis as well as a tumor antigen for targeted therapy.

Human GPC3 is a 70 kDa protein composed of 580 amino acid residues. It contains a Furin restriction site located between Arg358 and Cys359. GPC3 protein is cleaved at this site into two fragments: a 40 kDa N-terminal domain and a 30 kDa C-terminal domain, which are thereafter connected by one or more disulfide bonds. C-terminal residues Cys495 and Cys508 are modified with heparan sulfate, and residue Ser560 is linked to GPI.

Previous studies have demonstrated that GPC3 is highly associated with cancers including hepatocellular carcinoma (HCC). GPC3 is highly expressed in over 70% HCC patients, whereas its expression is not detected in liver cells in non-cancer patients such as hepatitis, cirrhosis, and fatty liver. Currently, the standard treatments with sorafenib, lenvatinib and regorafenib remain unsatisfactory in advanced HCC. GPC3, due to its high correlation with the occurrence and development of tumor including HCC, has become a promising therapeutic target for various cancer including liver cancer.

### SUMMARY OF THE INVENTION

The present disclosure provides novel antibodies binding to GPC3 or antigen binding fragments thereof, which can be in a form of a monoclonal antibody or bispecific antibody, such as a bispecific T-cell engager (BiTE). The antibodies disclosed herein are capable of binding to GPC3 and mediating killing of effector cells against target cells expressing GPC3 (such as various cancer cells).

In an aspect, the present disclosure provides an antibody specifically binding to GPC3, or an antigen binding fragment thereof, comprising a light chain variable region (VL) and a heavy chain variable region (VH), wherein (i) the VL comprises LCDRs 1-3 having the amino acid sequences as set forth in SEQ ID NOs: 1-3 respectively, and the VH comprises HCDRs 1-3 having the amino acid sequences as set forth in SEQ ID NOs: 6-8 respectively; or (ii) the VL comprises LCDRs 1-3 having the amino acid sequences as set forth in SEQ ID NOs: 23-25 respectively, and the VH comprises HCDRs 1-3 having the amino acid sequences as set forth in SEQ ID NOs: 60-62 respectively.

In some embodiments of the antibody or the antigen binding fragment thereof disclosed herein, (i) the VL comprises an amino acid sequence having at least 80%, at least 85%, at least 90%, at least 95%, at least 98%, at least 99%, or 100% sequence identity to SEQ ID NO: 4, and the VH comprises an amino acid sequence having at least 80%, at least 85%, at least 90%, at least 95%, at least 98%, at least 99%, or 100% sequence identity to SEQ ID NO: 9; or (ii) the VL comprises an amino acid sequence having at least 80%, at least 85%, at least 90%, at least 95%, at least 98%, at least 99%, or 100% sequence identity to SEQ ID NO: 26, and the VH comprises an amino acid sequence having at least 80%, at least 85%, at least 90%, at least 95%, at least 98%, at least 99%, or 100% sequence identity to SEQ ID NO: 28. In some embodiments, (i) the VL comprises an amino acid sequence as set forth in SEQ ID NO: 4 and the VH comprises an amino acid sequence as set forth in SEQ ID NO: 9; or (ii) the VL comprises an amino acid sequence as set forth in SEQ ID NO: 26 and the VH comprises an amino acid sequence as set forth in SEQ ID NO: 28.

In some embodiments, the antibody can be of an isotype selected from the group consisting of IgG, IgA, IgM, IgE and IgD. In some embodiments, the antibody can be of a subtype selected from the group consisting of IgG1, IgG2, IgG3, and IgG4.

In some embodiments, the antigen binding fragment can be selected from the group consisting of Fab, Fab′, F(ab′)₂, Fv, scFv, and ds-scFv.

In some embodiments, the antibody can be a monoclonal antibody. In some embodiments, the antibody comprises (i) a light chain comprising an amino acid sequence having at least 80%, at least 85%, at least 90%, at least 95%, at least 98%, at least 99%, or 100% sequence identity to SEQ ID NO: 5 and a heavy chain comprising an amino acid sequence having at least 80%, at least 85%, at least 90%, at least 95%, at least 98%, at least 99%, or 100% sequence identity to SEQ ID NO: 10; or (ii) a light chain comprising an amino acid sequence having at least 80%, at least 85%, at least 90%, at least 95%, at least 98%, at least 99%, or 100% sequence identity to SEQ ID NO: 27 and a heavy chain comprising an amino acid sequence having at least 80%, at least 85%, at least 90%, at least 95%, at least 98%, at least 99%, or 100% sequence identity to SEQ ID NO: 29.

In other embodiments, the antibody can be a bispecific or a multi-specific antibody. In some embodiments, the antibody is a bispecific antibody which further comprises a second antigen binding region binding to a second antigen. In some embodiments, the second antigen can be a tumor associated antigen or an immune cell antigen. In some embodiments, the second antigen is a T-cell antigen. In some embodiments, the T-cell antigen can be selected from the group consisting of T cell receptor (TCR), CD3, CD4, CD8, CD16, CD25, CD28, CD44, CD62L, CD69, ICOS, 41-BB (CD137), and NKG2D.

In some embodiments, the second antigen is CD3, and the second antigen binding region comprises a VL and a VH, wherein the VL comprises LCDRs 1-3 having the amino acid sequences as set forth in SEQ ID NOs: 11-13 respectively, and the VH comprises HCDRs 1-3 having the amino acid sequences as set forth in SEQ ID NOs: 16-18 respectively.

In some embodiments, the second antigen binding region comprises a VL comprising an amino acid sequence having at least 80%, at least 85%, at least 90%, at least 95%, at least 98%, at least 99%, or 100% sequence identity to SEQ ID NO: 14 and a VH comprising an amino acid sequence having at least 80%, at least 85%, at least 90%, at least 95%, at least 98%, at least 99%, or 100% sequence identity to SEQ ID NO: 19. In some embodiments, the second antigen binding region comprises a VL comprising an amino acid sequence as set forth in SEQ ID NO: 14 and a VH comprising an amino acid sequence as set forth in SEQ ID NO: 19.

In some embodiments, the VL of the second antigen binding region is linked to the C-terminal of the VL of the antibody specifically binding to GPC3, optionally via a first linker, and the VH of the second antigen binding region is linked to the C-terminal of the VH of the antibody specifically binding to GPC3, optionally via a second linker, wherein the first linker and the second linker are the same or different. In some embodiments, the first linker comprises an amino acid sequence as set forth in SEQ ID NO: 21 (GGGGSGGGGSGGGGS) or SEQ ID NO: 32 (GSGGGGSGGGGS), and the second linker comprises an amino acid sequence as set forth in SEQ ID NO: 22 (GGGSSGGGGSGGGGS).

In some embodiments, the bispecific antibody comprises (i) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20; or (ii) a light chain comprising an amino acid sequence having at least 80%, at least 85%, at least 90%, at least 95%, at least 98%, at least 99%, or 100% sequence identity to SEQ ID NO: 30 and a heavy chain comprising an amino acid sequence having at least 80%, at least 85%, at least 90%, at least 95%, at least 98%, at least 99%, or 100% sequence identity to SEQ ID NO: 31.

In some embodiments, the bispecific antibody can be a bispecific T-cell engager (BiTE).

In another aspect, the present disclosure provides a bispecific antibody or an antigen binding fragment thereof, comprising a first antigen binding region binding to GPC3 comprising a VL and a VH and a second antigen binding region binding to CD3 comprising a VL and a VH, wherein (i) the VL of the first antigen binding region comprises LCDRs 1-3 having the amino acid sequences as set forth in SEQ ID NOs: 1-3 respectively, and the VH of the first antigen binding region comprises HCDRs 1-3 having the amino acid sequences as set forth in SEQ ID NOs: 6-8 respectively; or (ii) the VL of the first antigen binding region comprises LCDRs 1-3 having the amino acid sequences as set forth in SEQ ID NOs: 23-25 respectively, and the VH of the first antigen binding region comprises HCDRs 1-3 having the amino acid sequences as set forth in SEQ ID NOs: 60-62 respectively; and the VL of the second antigen binding region comprises LCDRs 1-3 having the amino acid sequences as set forth in SEQ ID NOs: 11-13 respectively, and the VH of the second antigen binding region comprises HCDRs 1-3 having the amino acid sequences as set forth in SEQ ID NOs: 16-18 respectively.

In some embodiments of the bispecific antibody or the antigen binding fragment thereof disclosed herein, (i) the VL of the first antigen binding region comprises an amino acid sequence having at least 80%, at least 85%, at least 90%, at least 95%, at least 98%, at least 99%, or 100% sequence identity to SEQ ID NO: 4 and the VH of the first antigen binding region comprises an amino acid sequence having at least 80%, at least 85%, at least 90%, at least 95%, at least 98%, at least 99%, or 100% sequence identity to SEQ ID NO: 9; or (ii) the VL of the first antigen binding region comprises an amino acid sequence having at least 80%, at least 85%, at least 90%, at least 95%, at least 98%, at least 99%, or 100% sequence identity to SEQ ID NO: 26 and the VH of the first antigen binding region comprises an amino acid sequence having at least 80%, at least 85%, at least 90%, at least 95%, at least 98%, at least 99%, or 100% sequence identity to SEQ ID NO: 28; and the VL of the second antigen binding region comprises an amino acid sequence having at least 80%, at least 85%, at least 90%, at least 95%, at least 98%, at least 99%, or 100% sequence identity to SEQ ID NO: 14 and the VH of the second antigen binding region comprises an amino acid sequence having at least 80%, at least 85%, at least 90%, at least 95%, at least 98%, at least 99%, or 100% sequence identity to SEQ ID NO: 19.

In some embodiments, (i) the VL of the first antigen binding region comprises an amino acid sequence as set forth in SEQ ID NO: 4 and the VH of the first antigen binding region comprises an amino acid sequence as set forth in SEQ ID NO: 9; or (ii) the VL of the first antigen binding region comprises an amino acid sequence as set forth in SEQ ID NO: 26 and the VH of the first antigen binding region comprises an amino acid sequence as set forth in SEQ ID NO: 28; and the VL of the second antigen binding region comprises an amino acid sequence as set forth in SEQ ID NO: 14 and the VH of the second antigen binding region comprises an amino acid sequence as set forth in SEQ ID NO: 19.

In some embodiments, the VL of the second antigen binding region is linked to the C-terminal of the VL of the first antigen binding region, optionally via a first linker, and the VH of the second antigen binding region is linked to the C-terminal of the VH of the first antigen binding region, optionally via a second linker, wherein the first linker and the second linker are the same or different. In some embodiments, the first linker comprises an amino acid sequence as set forth in SEQ ID NO: 21 (GGGGSGGGGSGGGGS) or SEQ ID NO: 32 (GSGGGGSGGGGS), and the second linker comprises an amino acid sequence as set forth in SEQ ID NO: 22 (GGGSSGGGGSGGGGS).

In some embodiments, the bispecific antibody comprises (i) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20; or (ii) a light chain comprising an amino acid sequence having at least 80%, at least 85%, at least 90%, at least 95%, at least 98%, at least 99%, or 100% sequence identity to SEQ ID NO: 30 and a heavy chain comprising an amino acid sequence having at least 80%, at least 85%, at least 90%, at least 95%, at least 98%, at least 99%, or 100% sequence identity to SEQ ID NO: 31.

In some embodiments, the bispecific antibody can be a bispecific T-cell engager (BiTE).

In still another aspect, the present disclosure provides a nucleic acid comprising a nucleotide sequence encoding the antibody or the antigen binding fragment thereof disclosed herein or the bispecific antibody or the antigen binding fragment thereof disclosed herein.

In yet another aspect, the present disclosure provides a vector comprising the nucleic acid disclosed herein.

In another aspect, the present disclosure provides a host cell comprising the nucleic acid disclosed herein or the vector disclosed herein.

In still another aspect, the present disclosure provides a pharmaceutical composition comprising (i) the antibody or the antigen binding fragment thereof disclosed herein, or the bispecific antibody or the antigen binding fragment thereof disclosed herein; and (ii) a pharmaceutically acceptable carrier or excipient.

In some embodiments of the pharmaceutical composition disclosed herein, the pharmaceutical composition further comprises a second therapeutic agent. In some embodiments, the second therapeutic agent can be selected from an antibody, a chemotherapeutic agent and a small molecule drug. In some embodiments, the second therapeutic agent can be selected from a Bruton's tyrosine kinase (BTK) inhibitor, a PI3K inhibitor, a HDAC inhibitor, a PD-1/PD-L1 inhibitor, a LAG3 inhibitor, and glucocorticoid.

In yet another aspect, the present disclosure provides a conjugate comprising the antibody or the antigen binding fragment thereof disclosed herein or the bispecific antibody or the antigen binding fragment thereof disclosed herein, and a chemical moiety conjugated thereto.

In some embodiments of the conjugate disclosed herein, the chemical moiety is selected from the group consisting of a therapeutic agent, a detectable moiety, and an immune stimulatory molecule.

In another aspect, the present disclosure provides a method of treating a cancer in a subject comprising administering to the subject an effective amount of the antibody or the antigen binding fragment thereof disclosed herein, the bispecific antibody or the antigen binding fragment thereof disclosed herein, the pharmaceutical composition disclosed herein, or the conjugate disclosed herein.

In some embodiments of the method disclosed herein, the cancer is a GPC3 positive cancer. In some embodiments, the cancer is selected from the group consisting of liver cancer, colon cancer, pancreatic cancer, lung cancer, bladder cancer, melanoma and myeloma, preferably liver cancer or myeloma.

In some embodiments, the method further comprises administering to the subject a second therapeutic agent. In some embodiments, the second therapeutic agent is selected from an antibody, a chemotherapeutic agent and a small molecule drug. In some embodiments, the second therapeutic agent can be selected from a Bruton's tyrosine kinase (BTK) inhibitor, a PI3K inhibitor, a HDAC inhibitor, a PD-1/PD-L1 inhibitor, a LAG3 inhibitor, and glucocorticoid.

In still another aspect, the present disclosure provides a method of detecting GPC3 positive cancer in a subject comprising (i) contacting a sample obtained from the subject with the antibody or the antigen binding fragment thereof disclosed herein, the bispecific antibody or the antigen binding fragment thereof disclosed herein, or the conjugate disclosed herein; and (ii) detecting binding of the antibody or the antigen binding fragment thereof to GPC3 in the sample.

In some embodiments, the antibody or the antigen binding fragment thereof is linked to a detectable moiety. In some embodiments, the cancer is selected from the group consisting of liver cancer, colon cancer, pancreatic cancer, lung cancer, bladder cancer, melanoma and myeloma, preferably liver cancer or myeloma.

In yet another aspect, the present disclosure provides a kit for detecting the presence of a GPC3 antigen in a sample comprising the antibody or the antigen binding fragment thereof disclosed herein, the bispecific antibody or the antigen binding fragment thereof disclosed herein, or the conjugate disclosed herein. Preferably, the antibody or the antigen binding fragment thereof is linked to a detectable moiety.

In another aspect, the present disclosure provides use of the antibody or the antigen binding fragment thereof disclosed herein, the bispecific antibody or the antigen binding fragment thereof disclosed herein, the pharmaceutical composition disclosed herein, or the conjugate disclosed herein in the manufacture of a medicament for treating a cancer in a subject. In some embodiments, the cancer is a GPC3 positive cancer. In some embodiments, the cancer is selected from the group consisting of liver cancer, colon cancer, pancreatic cancer, lung cancer, bladder cancer, melanoma and myeloma, preferably liver cancer or myeloma.

In still another aspect, the present disclosure provides the antibody or the antigen binding fragment thereof disclosed herein, the bispecific antibody or the antigen binding fragment thereof disclosed herein, the pharmaceutical composition disclosed herein, or the conjugate disclosed herein for use in treating a cancer in a subject. In some embodiments, the cancer is a GPC3 positive cancer. In some embodiments, the cancer is selected from the group consisting of liver cancer, colon cancer, pancreatic cancer, lung cancer, bladder cancer, melanoma and myeloma, preferably liver cancer or myeloma.

In yet another aspect, the present disclosure provides use of the antibody or the antigen binding fragment thereof disclosed herein, the bispecific antibody or the antigen binding fragment thereof disclosed herein, or the conjugate disclosed herein in the manufacture of a kit for detecting GPC3 positive cancer in a subject. In some embodiments, the cancer is selected from the group consisting of liver cancer, colon cancer, pancreatic cancer, lung cancer, bladder cancer, melanoma and myeloma, preferably liver cancer or myeloma.

In still another aspect, the present disclosure provides the antibody or the antigen binding fragment thereof disclosed herein, the bispecific antibody or the antigen binding fragment thereof disclosed herein, or the conjugate disclosed herein for use in detecting GPC3 positive cancer in a subject. In some embodiments, the cancer is selected from the group consisting of liver cancer, colon cancer, pancreatic cancer, lung cancer, bladder cancer, melanoma and myeloma, preferably liver cancer or myeloma.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1A shows binding of 1A1 Fab against recombinant human GPC3 as measured by ELISA. BSA is used as negative control.

FIG. 1B shows binding of 6A4 Fab against recombinant human GPC3 as measured by ELISA. BSA is used as negative control.

FIG. 2A shows binding of 1A1 Fab against tumor cell lines Huh7, HepG2 and SK-HEP-1 as measured by flow cytometry. A commercial anti-GPC3 antibody (GPC3-PE) is used as positive control. Color code, purple: negative control: green: 1A1 Fab or GPC3-PE antibody.

FIG. 2B shows binding of 6A4 Fab against tumor cell lines Huh7 and A549 as measured by flow cytometry. A commercial anti-GPC3 antibody (GPC3-PE) is used as positive control. Color code, purple: negative control: green: 6A4 Fab.

FIG. 3A shows binding of 1A1 mAb against recombinant human, cynomolgus and mouse GPC3, as measured by ELISA. BSA is used as negative control.

FIG. 3B shows binding of 6A4 mAb against recombinant human GPC3, as measured by ELISA.

FIG. 4A shows binding of 1A1 mAb to tumor cell lines HepG2, HuH7, RPMI8226, H226 and SK-HEP-1, as measured by flow cytometry. Color code, purple: negative control: green: 1A1 mAb.

FIG. 4B shows binding of 6A4 mAb to tumor cell line HepG2 as measured by flow cytometry. An IgG isotype antibody is used as negative control.

FIG. 5A shows binding of 1A1-based GPC3×CD3 HBiTE against recombinant human, cynomolgus and mouse GPC3, as measured by ELISA. BSA is used as negative control.

FIG. 5B shows binding of 1A1-based GPC3×CD3 HBiTE against recombinant human CD3, as measured by ELISA.

FIG. 5C shows binding of 6A4-based GPC3×CD3 HBiTE against recombinant human GPC3, as measured by ELISA.

FIG. 5D shows binding of 6A4-based GPC3×CD3 HBiTE against recombinant human CD3, as measured by ELISA.

FIG. 6A shows binding of 1A1-based GPC3×CD3 HBITE to tumor cell lines HepG2, Huh7, RPMI8226, A375, and 5637, as well as Jurkat cells (CD3 positive), as measured by flow cytometry. Color code, purple: negative control: green: 1A1 HBITE.

FIG. 6B shows binding of 6A4-based GPC3×CD3 HBiTE to tumor cell lines HepG2, Huh7 and RPMI8226, as measured by flow cytometry. Color code, purple: negative control: green: 6A4 HBiTE.

FIG. 7 shows killing of 1A1-based GPC3×CD3 HBiTE against Hep-G2 cells in the presence of human PBMC. The ratio of target cells (Hep-G2) to effector cells (PBMC) is 1:5.

FIG. 8 shows killing of 1A1-based GPC3×CD3 HBiTE against HuH7 cells in the presence of human PBMC. The ratio of target cells (HuH7) to effector cells (PBMC) is 1:5.

FIG. 9A shows images of RPMI8226 tumor cell clusters after treating with 1A1-based GPC3×CD3 HBiTE at indicated concentrations in the presence of human PBMC. The ratio of target cells (RPMI8226) to effector cells (PBMC) is 1:5.

FIG. 9B shows killing of 1A1-based GPC3×CD3 HBiTE against RPMI8226 cells in the presence of human PBMC. The ratio of target cells (RPMI8226) to effector cells (PBMC) is 1:5.

FIG. 10A shows images of LS174T-GPC3 tumor cell clusters after treating with 1A1-based GPC3×CD3 HBiTE at indicated concentrations in the presence of human PBMC. The ratio of target cells (LS174T-GPC3) to effector cells (PBMC) is 1:5.

FIG. 10B shows killing of 1A1-based GPC3×CD3 HBITE against LS174T-GPC3 cells in the presence of human PBMC. The ratio of target cells (LS174T-GPC3) to effector cells (PBMC) is 1:5.

FIG. 11 shows killing of 6A4-based GPC3×CD3 HBiTE against HuH7 cells in the presence of human PBMC. The ratio of target cells (HuH7) to effector cells (PBMC) is 1:12.5.

FIG. 12 shows inhibition of 1A1-based GPC3×CD3 HBiTE against tumors derived from LS174T-GPC3 cells in a mouse model.

FIG. 13 shows inhibition of 1A1-based GPC3×CD3 HBiTE against tumors derived from Huh-7 cells in a mouse model.

FIG. 14 shows inhibition of 6A4-based GPC3×CD3 HBiTE against tumors derived from LS174T-GPC3 cells in a mouse model.

FIG. 15A shows images of ADCC killing of 1A1 mAb and 6A4 mAb against HepG2 cells in the presence of NK cells.

FIG. 15B shows ADCC killing of 1A1 mAb and 6A4 mAb against HepG2 cells in the presence of NK cells.

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland: Sambrook et al, “Molecular Cloning: A Laboratory Manual” (2nd Ed.), Vols. 1-3, Cold Spring Harbor Laboratory Press (1989): F. Ausubel et al, eds., “Current protocols in molecular biology”, Green Publishing and Wiley InterScience, New York (1987): Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

Unless indicated or defined otherwise, the term “comprise”, and variations such as “comprises” and “comprising”, should be understood to imply the inclusion of a stated elements or step or group of elements or steps but not the exclusion of any other element or step or group of elements or steps.

As used herein, the term “antibody” refers to an immunoglobulin molecule which has the ability to specifically bind to a specific antigen. An antibody often comprises a variable region and a constant region in each of a heavy chain and a light chain. The variable regions of the heavy and light chains of antibodies contain a binding domain that interacts with an antigen. The constant regions of antibodies may mediate the binding of the immunoglobulin to host tissues or factors, including various cells of the immune system (such as effector cells) and components of the complement system such as CIq, the first component in the classical pathway of complement activation. Accordingly, most antibodies have a heavy chain variable region (VH) and a light chain variable region (VL) that together form the portion of the antibody that binds to the antigen.

A “light chain variable region” (VL) or “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989): a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering system) in which corresponding residues between different heavy chains or between different light chains are assigned the same number. The present disclosure can use CDRs defined according to any of these numbering systems, although preferred embodiments use Kabat defined CDRs.

The term “antibody” as used herein should be understood in its broadest meaning, and includes monoclonal antibodies (including full-length monoclonal antibodies), polyclonal antibodies, antibody fragments, and multi-specific antibodies containing at least two different antigen binding regions (e.g., bispecific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

As used herein, the term “antigen binding fragment” of an antibody refers to one or more fragments of an antibody that retain the ability to specifically bind to an antigen (e.g., a GPC3 protein). It has been shown that the antigen binding function of an antibody can be performed by fragments of a full-length antibody.

Examples of antigen binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3.sup.rd ed. 1993); (iv) a Fd fragment consisting of the VH and CH1 domains; (v) a Fd′ fragment having VH and CH1 domains and one or more cysteine residues at the C-terminus of the CH1 domain; (vi) a Fv fragment consisting of the VL and VH domains of a single arm of an antibody, (vii) a dAb fragment (Ward et al., (1989) Nature 341:544-546), which consists of a VH domain; (viii) an isolated complementarity determining region (CDR); and (ix) a nanobody, a heavy chain variable region containing a single variable domain and two constant domains. Furthermore, although the two domains of the Fv fragment, V Land VH are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. Furthermore, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity.

These antigen binding fragments can be obtained using conventional techniques known to those with skill in the art, and the fragments are screened for utility in the same manner as are intact antibodies.

As used herein, the term “binding” or “specifically binding” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant and an antigen-binding site on the antibody: the lesser the value of the KD, the stronger the binding strength between an antigenic determinant and the antibody. Alternatively, the affinity can also be expressed as the affinity constant (KA), which is 1/KD.

Avidity is the measure of the strength of binding between an antibody and the pertinent antigen. Avidity is related to both the affinity between an antigenic determinant and its antigen binding site on the antibody and the number of pertinent binding sites present on the antibody. Typically, an antibody will bind with a dissociation constant (KD) of 10⁻⁵ to 10⁻¹² M or less, and preferably 10⁻⁷ to 10⁻¹² M or less and more preferably 10⁻⁸ to 10⁻¹² M, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ M is generally considered to indicate non-specific binding. Specifically binding of an antibody to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y.

Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13:211, 1974 and Adv. Enzymol., 47:45-149, 1978, and on the analysis of hydrophobicity patterns in proteins developed by Eisenberg et al., Proc. Nat. Acad Sci. USA 81:140-144, 1984: Kyte & Doolittle, J Mol. Biol. 157:105-132, 198 1, and Goldman et al., Ann. Rev. Biophys. Chem. 15:321-353, 1986, all incorporated herein in their entirety by reference.

As used herein, the term “monoclonal antibody” refers to an antibody obtained from a substantially homogeneous antibody population. That is, each antibodies constituting the population are the same, except for possible naturally occurring mutations in small amount. Monoclonal antibodies are highly specific and are directed against a single antigen. The term “monoclonal antibody” herein is not limited to antibodies produced by hybridoma technology, and should not be interpreted as requiring production of antibodies by any specific method.

The term “bispecific antibody” is in the context of the present invention to be understood as an antibody having two different antigen-binding regions defined by different antibody sequences. This can be understood as different target binding but includes as well binding to different epitopes in one target.

As used herein, the term “tumor associated antigen” refers to an antigen that is differentially expressed in cancer cells compared to normal cells, and therefore can be used to target cancer cells.

As used herein, the term “CD3” refers to the human CD3 protein complex, which has five peptide chains, γ chain, δ chain, ε chain, ξ chain and η chain, and is associated with the T cell receptor α and β chain to form a TCR-CD3 complex. The term includes any CD3 variants, isoforms and species homologs which are naturally expressed by cells, including T cells, or are expressed on cells transfected with genes or cDNA encoding the aforementioned chains.

As used herein, the term “bispecific T-cell engager” or “BiTE” refers to single polypeptide chain molecules that having two antigen-binding domains, one of which binds to a T-cell antigen and the second of which binds to an antigen present on the surface of a target (See, PCT Publication WO 05/061547; Baeuerle et al., 2008, Drugs of the Future 33:137-147; Bargou, et al., 2008, Science 321:974-977, which are incorporated herein by reference in their entireties). Thus, the BiTE of the disclosure has an antigen binding region that binds to GPC3 and a second antigen binding region that is directed towards a T-cell antigen.

As used herein, the term “vector” is intended to refer to a nucleic acid molecule capable of transporting another nucleic acid to which it has been linked.

As used herein, the term “host cell” refers to a cell into which an expression vector has been introduced.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g. cancers). The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment, or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc.

In an aspect, the present disclosure provides an antibody specifically binding to GPC3, or an antigen binding fragment thereof, comprising a light chain variable region (VL) and a heavy chain variable region (VH), wherein (i) the VL comprises LCDRs 1-3 having the amino acid sequences as set forth in SEQ ID NOs: 1-3 respectively, and the VH comprises HCDRs 1-3 having the amino acid sequences as set forth in SEQ ID NOs: 6-8 respectively; or (ii) the VL comprises LCDRs 1-3 having the amino acid sequences as set forth in SEQ ID NOs: 23-25 respectively, and the VH comprises HCDRs 1-3 having the amino acid sequences as set forth in SEQ ID NOs: 60-62 respectively.

In some embodiments, CDR sequences are defined according to Kabat numbering system.

When CDR sequences are defined according to Kabat numbering system, the VL of the antibody disclosed herein comprises LCDR1, LCDR2 and LCDR3 having the amino acid sequences as set forth in SEQ ID NO: 1 (RSSQSLVYSDGNTYLN), SEQ ID NO: 2 (KVSNRDS) and SEQ ID NO: 3 (MQSTHWPLT) respectively, and the VH of the antibody disclosed herein comprises HCDR1, HCDR2 and HCDR3 having the amino acid sequences as set forth in SEQ ID NO: 6 (SYGIS), SEQ ID NO: 7 (WISAYNGNTNYAQKLQG) and SEQ ID NO: 8 (AGTPTQILRYFDWLSQPFDY) respectively; or the VL of the antibody disclosed herein comprises LCDR1, LCDR2 and LCDR3 having the amino acid sequences as set forth in SEQ ID NO: 23 (RASQSISSYLN), SEQ ID NO: 24 (AASSLQS) and SEQ ID NO: 25 (QQSYSTPLT) respectively, and the VH of the antibody disclosed herein comprises HCDR1, HCDR2 and HCDR3 having the amino acid sequences as set forth in SEQ ID NO: 60 (SYAMH), SEQ ID NO: 61 (WINAGNGNTKYSQKFQG) and SEQ ID NO: 62 (DPSH) respectively.

In some embodiments of the antibody or the antigen binding fragment thereof disclosed herein, (i) the VL comprises an amino acid sequence having at least 80%, at least 85%, at least 90%, at least 95%, at least 98%, at least 99%, or 100% sequence identity to SEQ ID NO: 4, and the VH comprises an amino acid sequence having at least 80%, at least 85%, at least 90%, at least 95%, at least 98%, at least 99%, or 100% sequence identity to SEQ ID NO: 9; or (ii) the VL comprises an amino acid sequence having at least 80%, at least 85%, at least 90%, at least 95%, at least 98%, at least 99%, or 100% sequence identity to SEQ ID NO: 26, and the VH comprises an amino acid sequence having at least 80%, at least 85%, at least 90%, at least 95%, at least 98%, at least 99%, or 100% sequence identity to SEQ ID NO: 28.

In some embodiments, the VL comprises a functional variant of the amino acid sequence as set forth in SEQ ID NO: 4 or SEQ ID NO: 26 formed by insertion, deletion and/or substitution of one or more amino acid(s) therein, provided that the functional variant retains the ability of binding to GPC3. In some embodiments, the VH comprises a functional variant of the amino acid sequence as set forth in SEQ ID NO: 9 or SEQ ID NO: 28 formed by insertion, deletion and/or substitution of one or more amino acid(s) therein, provided that the functional variant retains the ability of binding to GPC3.

The functional variant comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the amino acid sequence of the parent polypeptide. For example, the functional variant of SEQ ID NO: 4 or SEQ ID NO: 26 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 4 or SEQ ID NO: 26. For example, the functional variant of SEQ ID NO: 9 or SEQ ID NO: 28 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 9 or SEQ ID NO: 28.

In some embodiments, the functional variant of SEQ ID NO: 4 or SEQ ID NO: 26 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 4 or SEQ ID NO: 26 and formed by insertion, deletion and/or substitution of one or more amino acid(s) in SEQ ID NO: 4 or SEQ ID NO: 26. In some embodiments, the functional variant of SEQ ID NO: 9 or SEQ ID NO: 28 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 9 or SEQ ID NO: 28 and formed by insertion, deletion and/or substitution of one or more amino acid(s) in SEQ ID NO: 9 or SEQ ID NO: 28.

In the context of the functional variant, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In a preferred embodiment, the VL comprises an amino acid sequence as set forth in SEQ ID NO: 4 and the VH comprises an amino acid sequence as set forth in SEQ ID NO: 9; or the VL comprises an amino acid sequence as set forth in SEQ ID NO: 26 and the VH comprises an amino acid sequence as set forth in SEQ ID NO: 28.

Based on the amino acid sequence of heavy chain constant regions of the antibody, a immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (2) chain or a kappa (κ) chain, based on the amino acid sequence of the light chain. The antibodies disclosed herein can be of any classes or subtypes above.

In some embodiments, the antibody can be of an isotype selected from the group consisting of IgG, IgA, IgM, IgE and IgD. In some embodiments, the antibody can be of a subtype selected from the group consisting of IgG1, IgG2, IgG3, and IgG4. In a preferred embodiment, the antibody is an IgG1 antibody.

The antibody disclosed herein can be an intact antibody or the antigen binding fragment thereof. The antigen binding fragment can be any fragments of the antibody that retain the ability to specifically bind to GPC3. Examples of antigen binding fragments include but are not limited to a Fab fragment; a F(ab′)2 fragment; a Fab′ fragment; a Fd fragment; a Fd′ fragment; a Fv fragment; a scFv fragment; a dAb fragment; an isolated complementarity determining region (CDR): a nanobody: a linear antibody comprising a pair of tandem Fd segments (VH-CH1-VH-CH1), and a modified version of any of the foregoing fragments, which retains antigen binding activity.

In some embodiments, the antigen binding fragment can be selected from the group consisting of Fab, Fab′, F(ab′)₂, Fv, scFv, and ds-scFv. In a preferred embodiment, the antigen binding fragment is Fab or scFv.

In some embodiments, the antibody can be a monoclonal antibody. In some embodiments, the antibody comprises (i) a light chain comprising an amino acid sequence having at least 80%, at least 85%, at least 90%, at least 95%, at least 98%, at least 99%, or 100% sequence identity to SEQ ID NO: 5 and a heavy chain comprising an amino acid sequence having at least 80%, at least 85%, at least 90%, at least 95%, at least 98%, at least 99%, or 100% sequence identity to SEQ ID NO: 10; or (ii) a light chain comprising an amino acid sequence having at least 80%, at least 85%, at least 90%, at least 95%, at least 98%, at least 99%, or 100% sequence identity to SEQ ID NO: 27 and a heavy chain comprising an amino acid sequence having at least 80%, at least 85%, at least 90%, at least 95%, at least 98%, at least 99%, or 100% sequence identity to SEQ ID NO: 29.

In some embodiments, the light chain comprises a functional variant of the amino acid sequence as set forth in SEQ ID NO: 5 or SEQ ID NO: 27 formed by insertion, deletion and/or substitution of one or more amino acid(s) therein, provided that the functional variant retains the ability of binding to GPC3. In some embodiments, the heavy chain comprises a functional variant of the amino acid sequence as set forth in SEQ ID NO: 10 or SEQ ID NO: 29 formed by insertion, deletion and/or substitution of one or more amino acid(s) therein, provided that the functional variant retains the ability of binding to GPC3.

For example, the functional variant of SEQ ID NO: 5 or SEQ ID NO: 27 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 5 or SEQ ID NO: 27. For example, the functional variant of SEQ ID NO: 10 or SEQ ID NO: 29 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 10 or SEQ ID NO: 29.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-50, preferably 1-20, more preferably 1-10, still more preferably 1-5. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3 and/or FR4; and/or constant regions, e.g., CL, CH1, CH2 and/or CH3.

In some embodiments, the substitution of one or more amino acid(s) can be conservative substitution of one or more amino acid(s). Examples of conservative substitutions are as described above.

In a preferred embodiment, the light chain comprises an amino acid sequence as set forth in SEQ ID NO: 5 and the heavy chain comprises an amino acid sequence as set forth in SEQ ID NO: 10; or the light chain comprises an amino acid sequence as set forth in SEQ ID NO: 27 and the heavy chain comprises an amino acid sequence as set forth in SEQ ID NO: 29.

In other embodiments, the antibody can be a bispecific or a multi-specific antibody. In some embodiments, the antibody is a bispecific antibody which further comprises a second antigen binding region binding to a second antigen. In some embodiments, the second antigen can be a tumor associated antigen or an immune cell antigen.

Many tumor associated antigens associated with specific cancers have been identified in the art. In some embodiments, tumor-associated antigens are antigens that can potentially stimulate an obvious tumor-specific immune response. Some of these antigens are encoded by normal cells, but not necessarily expressed by normal cells. These antigens can be characterized as those that are usually silent (i.e., not expressed) in normal cells, those that are expressed only during certain stages of differentiation, and those that are expressed over time, such as embryonic and fetal antigens. Other cancer antigens are encoded by mutant cell genes such as oncogenes (e.g. activated ras oncogene), suppressor genes (e.g. mutant p53), and fusion proteins produced by internal deletions or chromosomal translocations. Other cancer antigens can be encoded by viral genes, such as those carried on RNA and DNA tumor viruses. Many other tumor associated antigens and antibodies against them are known and/or commercially available, and can also be produced by those skilled in the art.

Examples of tumor associated antigens include but are not limited to 5T4, alphafetoprotein, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFR, EGFRVIII, epithelial tumor antigen, ERBB2, FLT3, folate binding protein, GD2, GD3, HIV-1 envelope glycoprotein gp41, HIV-1 envelope glycoprotein gpl20, melanoma-associated antigen, mesothelin, MUC-1, mutated p53, mutated ras, ROR1, VEGFR2, and combinations thereof.

In some embodiments, the second antigen is a T-cell antigen. In some embodiments, the T-cell antigen can be selected from the group consisting of T cell receptor (TCR), CD3, CD4, CD8, CD16, CD25, CD28, CD44, CD62L, CD69, ICOS, 41-BB (CD137), and NKG2D or any combination thereof. 8, CD44, CD62L, CD69, ICOS, 41-BB (CD137), and NKG2D or any combination thereof. In some embodiments, the T-cell antigen is CD3, and the second antigen binding region binds to any of γ chain, δ chain, ε chain, ε chain and η chain of CD3.

In some embodiments, the second antigen is CD3, and the second antigen binding region comprises a VL and a VH, wherein the VL comprises LCDRs 1-3 having the amino acid sequences as set forth in SEQ ID NOs: 11-13 respectively, and the VH comprises HCDRs 1-3 having the amino acid sequences as set forth in SEQ ID NOs: 16-18 respectively.

In some embodiments, CDR sequences are defined according to Kabat numbering system. When using Kabat defined CDR sequences, the VL of the second antigen binding region disclosed herein comprises LCDR1, LCDR2 and LCDR3 having the amino acid sequences as shown in SEQ ID NO: 11 (RSSTGAVTTSNYAN), SEQ ID NO: 12 (GANKRAP) and SEQ ID NO: 13 (ALWYSNLWV) respectively, and the VH of the second antigen binding region disclosed herein comprises HCDR1, HCDR2 and HCDR3 having the amino acid sequences as shown in SEQ ID NO: 16 (GFTFNTY), SEQ ID NO: 17 (RSKYNNYA) and SEQ ID NO: 18 (HGNFGSSYVSYFAY) respectively.

In some embodiments, the second antigen binding region comprises a VL comprising an amino acid sequence having at least 80%, at least 85%, at least 90%, at least 95%, at least 98%, at least 99%, or 100% sequence identity to SEQ ID NO: 14 and a VH comprising an amino acid sequence having at least 80%, at least 85%, at least 90%, at least 95%, at least 98%, at least 99%, or 100% sequence identity to SEQ ID NO: 19.

In some embodiments, the VL comprises a functional variant of the amino acid sequence as set forth in SEQ ID NO: 14 formed by insertion, deletion and/or substitution of one or more amino acid(s) therein, provided that the functional variant retains the ability of binding to CD3. In some embodiments, the VH comprises a functional variant of the amino acid sequence as set forth in SEQ ID NO: 19 formed by insertion, deletion and/or substitution of one or more amino acid(s) therein, provided that the functional variant retains the ability of binding to CD3.

For example, the functional variant of SEQ ID NO: 14 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 14. For example, the functional variant of SEQ ID NO: 19 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 19.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Examples of conservative substitutions are as described above.

In a preferred embodiment, the second antigen binding region comprises a VL comprising an amino acid sequence as set forth in SEQ ID NO: 14 and a VH comprising an amino acid sequence as set forth in SEQ ID NO: 19.

In some embodiments, the VL of the second antigen binding region is linked to the C-terminal of the VL of the antibody specifically binding to GPC3, optionally via a first linker, and the VH of the second antigen binding region is linked to the C-terminal of the VH of the antibody specifically binding to GPC3, optionally via a second linker, wherein the first linker and the second linker are the same or different. In some embodiments, the first linker comprises an amino acid sequence as set forth in SEQ ID NO: 21 (GGGGSGGGGSGGGGS) or SEQ ID NO: 32 (GSGGGGSGGGGS), and the second linker comprises an amino acid sequence as set forth in SEQ ID NO: 22 (GGGSSGGGGSGGGGS).

In some embodiments, the bispecific antibody comprises (i) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20; or (ii) a light chain comprising an amino acid sequence having at least 80%, at least 85%, at least 90%, at least 95%, at least 98%, at least 99%, or 100% sequence identity to SEQ ID NO: 30 and a heavy chain comprising an amino acid sequence having at least 80%, at least 85%, at least 90%, at least 95%, at least 98%, at least 99%, or 100% sequence identity to SEQ ID NO: 31.

In some embodiments, the light chain comprises a functional variant of the amino acid sequence as set forth in SEQ ID NO: 15 or SEQ ID NO: 30 formed by insertion, deletion and/or substitution of one or more amino acid(s) therein, provided that the functional variant retains the ability of binding to GPC3 and CD3. In some embodiments, the heavy chain comprises a functional variant of the amino acid sequence as set forth in SEQ ID NO: 20 or SEQ ID NO: 31 formed by insertion, deletion and/or substitution of one or more amino acid(s) therein, provided that the functional variant retains the ability of binding to GPC3 and CD3.

For example, the functional variant of SEQ ID NO: 15 or SEQ ID NO: 30 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 15 or SEQ ID NO: 30. For example, the functional variant of SEQ ID NO: 20 or SEQ ID NO: 31 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 20 or SEQ ID NO: 31.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-50, preferably 1-20, more preferably 1-10, still more preferably 1-5. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3 and/or FR4; and/or constant regions, e.g., CL, CH1, CH2 and/or CH3.

In some embodiments, the substitution of one or more amino acid(s) can be conservative substitution of one or more amino acid(s). Examples of conservative substitutions are as described above.

In a preferred embodiment, the light chain comprises an amino acid sequence as set forth in SEQ ID NO: 15 and the heavy chain comprises an amino acid sequence as set forth in SEQ ID NO: 20; or the light chain comprises an amino acid sequence as set forth in SEQ ID NO: 30 and the heavy chain comprises an amino acid sequence as set forth in SEQ ID NO: 31.

In some embodiments, the bispecific antibody can be a bispecific T-cell engager (BiTE). In some embodiments of the antibody or the antigen binding fragment thereof disclosed herein, the bispecific antibody is in form of an HBITE as described in PCT application No. PCT/US2018/016524 (which is incorporated herein by reference in its entirety). In the HBITE, the light chain, from N-terminus to C-terminus, comprises an anti-target VL domain, an anti-CD3 VL-CL and a monomeric human IgG1 Fc (e.g., mFc7.2); and the heavy chain, from N-terminus to C-terminus, comprises an anti-target VH domain, an anti-CD3 VH-CH1 and a monomeric human IgG1 Fc (e.g., mFc7.2). Monomeric Fc7.2 contains two amino acid mutations (T366L and Y407H) capable of inhibiting Fc homodimerization.

In another aspect, the present disclosure provides a bispecific antibody or an antigen binding fragment thereof, comprising a first antigen binding region binding to GPC3 comprising a VL and a VH and a second antigen binding region binding to CD3 comprising a VL and a VH, wherein (i) the VL of the first antigen binding region comprises LCDRs 1-3 having the amino acid sequences as set forth in SEQ ID NOs: 1-3 respectively, and the VH of the first antigen binding region comprises HCDRs 1-3 having the amino acid sequences as set forth in SEQ ID NOs: 6-8 respectively; or (ii) the VL of the first antigen binding region comprises LCDRs 1-3 having the amino acid sequences as set forth in SEQ ID NOs: 23-25 respectively, and the VH of the first antigen binding region comprises HCDRs 1-3 having the amino acid sequences as set forth in SEQ ID NOs: 60-62 respectively; and the VL of the second antigen binding region comprises LCDRs 1-3 having the amino acid sequences as set forth in SEQ ID NOs: 11-13 respectively, and the VH of the second antigen binding region comprises HCDRs 1-3 having the amino acid sequences as set forth in SEQ ID NOs: 16-18 respectively.

In some embodiments of the bispecific antibody or the antigen binding fragment thereof disclosed herein, (i) the VL of the first antigen binding region comprises an amino acid sequence having at least 80%, at least 85%, at least 90%, at least 95%, at least 98%, at least 99%, or 100% sequence identity to SEQ ID NO: 4 and the VH of the first antigen binding region comprises an amino acid sequence having at least 80%, at least 85%, at least 90%, at least 95%, at least 98%, at least 99%, or 100% sequence identity to SEQ ID NO: 9; or (ii) the VL of the first antigen binding region comprises an amino acid sequence having at least 80%, at least 85%, at least 90%, at least 95%, at least 98%, at least 99%, or 100% sequence identity to SEQ ID NO: 26 and the VH of the first antigen binding region comprises an amino acid sequence having at least 80%, at least 85%, at least 90%, at least 95%, at least 98%, at least 99%, or 100% sequence identity to SEQ ID NO: 28; and the VL of the second antigen binding region comprises an amino acid sequence having at least 80%, at least 85%, at least 90%, at least 95%, at least 98%, at least 99%, or 100% sequence identity to SEQ ID NO: 14 and the VH of the second antigen binding region comprises an amino acid sequence having at least 80%, at least 85%, at least 90%, at least 95%, at least 98%, at least 99%, or 100% sequence identity to SEQ ID NO: 19.

In some embodiments, the VL of the first antigen binding region comprises a functional variant of the amino acid sequence as set forth in SEQ ID NO: 4 or SEQ ID NO: 26 formed by insertion, deletion and/or substitution of one or more amino acid(s) therein, provided that the functional variant retains the ability of binding to GPC3. In some embodiments, the VH of the first antigen binding region comprises a functional variant of the amino acid sequence as set forth in SEQ ID NO: 9 or SEQ ID NO: 28 formed by insertion, deletion and/or substitution of one or more amino acid(s) therein, provided that the functional variant retains the ability of binding to GPC3. In some embodiments, the VL of the second antigen binding region comprises a functional variant of the amino acid sequence as set forth in SEQ ID NO: 14 formed by insertion, deletion and/or substitution of one or more amino acid(s) therein, provided that the functional variant retains the ability of binding to CD3. In some embodiments, the VH of the second antigen binding region comprises a functional variant of the amino acid sequence as set forth in SEQ ID NO: 19 formed by insertion, deletion and/or substitution of one or more amino acid(s) therein, provided that the functional variant retains the ability of binding to CD3.

The functional variants of SEQ ID NOs: 4, 9, 14, 19, 26 and 28 can be those as described above.

In a preferred embodiment, the VL of the first antigen binding region comprises an amino acid sequence as set forth in SEQ ID NO: 4 and the VH of the first antigen binding region comprises an amino acid sequence as set forth in SEQ ID NO: 9; or the VL of the first antigen binding region comprises an amino acid sequence as set forth in SEQ ID NO: 26 and the VH of the first antigen binding region comprises an amino acid sequence as set forth in SEQ ID NO: 28; and the VL of the second antigen binding region comprises an amino acid sequence as set forth in SEQ ID NO: 14 and the VH of the second antigen binding region comprises an amino acid sequence as set forth in SEQ ID NO: 19.

In some embodiments, the VL of the second antigen binding region is linked to the C-terminal of the VL of the first antigen binding region, optionally via a first linker, and the VH of the second antigen binding region is linked to the C-terminal of the VH of the first antigen binding region, optionally via a second linker, wherein the first linker and the second linker are the same or different. In some embodiments, the first linker comprises an amino acid sequence as set forth in SEQ ID NO: 21 (GGGGSGGGGSGGGGS) or SEQ ID NO: 32 (GSGGGGSGGGGS), and the second linker comprises an amino acid sequence as set forth in SEQ ID NO: 22 (GGGSSGGGGSGGGGS).

In some embodiments, the bispecific antibody comprises a single polypeptide chain comprising the first antigen binding region and the second antigen binding region, and optionally an Fc region.

The Fc region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fc region is human IgG1 Fc.

In one embodiment, the Fc region is effector-function-deficient. For example, the Fc region may be of an IgG1 isotype, or a non-IgG1 type, e.g. IgG2, IgG3 or IgG4, which has been mutated such that the ability to mediate effector functions, such as ADCC, has been reduced or even eliminated. Such mutations have e.g. been described in Dall'Acqua W F et al., J Immunol. 177 (2): 1129-1138 (2006) and Hezareh M, J Virol.: 75 (24): 12161-12168 (2001).

In one embodiment, the Fc region comprises a mutation removing the acceptor site for Asn-linked glycosylation or is otherwise manipulated to change the glycosylation properties. For example, in an IgG1 Fc region, an N297Q mutation can be used to remove an Asn-linked glycosylation site. Accordingly, in a specific embodiment, Fc region comprise an IgG1 wildtype sequence with an N297Q mutation.

In a further embodiment, the Fc region is glyco-engineered to reduce fucose and thus enhance ADCC, e.g. by addition of compounds to the culture media during antibody production as described in US2009317869 or as described in van Berkel et al. (2010) Biotechnol. Bioeng. 105:350 or by using FUT8 knockout cells, e.g. as described in Yamane-Ohnuki et al. (2004) Biotechnol. Bioeng 87:614. ADCC may alternatively be optimized using the method described by Umaña et al. (1999) Nature Biotech 17:176. In a further embodiment, the Fc region has been engineered to enhance complement activation, e.g. as described in Natsume et al. (2009) Cancer Sci. 100:2411.

In some embodiments, the Fc region comprises modifications or mutations that can inhibit Fc homodimerization. In some embodiments, the Fc region comprises a variant of a human IgG1 Fc wildtype sequence. The variant can comprise amino acid substitutions at positions T366 and Y407 of human IgG1 (Kabat numbering). Preferably, T366 is substituted with L (Leucine). Preferably, Y407 is substituted with I (Isoleucine), F (Phenylalanine), L (Leucine), M (Methionine), H (Histidine), K (Lysine), S (Serine), Q (Glutamine), T (Threonine), W (Tryptophan), A (Alanine), G (Glycine) or N (Asparagine). More preferably, Y407 is substituted with H. In one embodiment, T366 is substituted with L, and Y407 is substituted with H.

In some embodiments, the Fc region can be a monomeric human IgG1 Fc (e.g., mFc7.2) as described in PCT application No. PCT/US2018/016524, which is incorporated herein by reference in its entirety.

In some embodiments, the bispecific antibody comprises a first polypeptide chain comprising the VL of the first antigen binding region and the VL of the second antigen binding region, and optionally an Fc region; and a second polypeptide chain comprising the VH of the first antigen binding region and the VH of the second antigen binding region, and optionally an Fc region. The Fc region can be those as describe above.

In some embodiments, the first polypeptide chain further comprises a light chain constant region (CL). In some embodiments, the first polypeptide chain comprises a monomeric human IgG1 Fc (e.g., mFc7.2) as described above. In some embodiments, the first polypeptide chain comprises, from N-terminal to C-terminal: the VL of the first antigen binding region, the VL of the second antigen binding region, CL and mFc7.2.

In some embodiments, the second polypeptide chain further comprises a heavy chain constant region (CH), e.g., CH1. In some embodiments, the first polypeptide chain comprises a monomeric human IgG1 Fc (e.g., mFc7.2) as described above. In some embodiments, the second polypeptide chain comprises, from N-terminal to C-terminal: the VH of the first antigen binding region, the VH of the second antigen binding region, CH1 and mFc7.2.

In some embodiments, the bispecific antibody comprises (i) a light chain comprising an amino acid sequence having at least 80%, at least 85%, at least 90%, at least 95%, at least 98%, at least 99%, or 100% sequence identity to SEQ ID NO: 15 and a heavy chain comprising an amino acid sequence having at least 80%, at least 85%, at least 90%, at least 95%, at least 98%, at least 99%, or 100% sequence identity to SEQ ID NO: 20; or (ii) a light chain comprising an amino acid sequence having at least 80%, at least 85%, at least 90%, at least 95%, at least 98%, at least 99%, or 100% sequence identity to SEQ ID NO: 30 and a heavy chain comprising an amino acid sequence having at least 80%, at least 85%, at least 90%, at least 95%, at least 98%, at least 99%, or 100% sequence identity to SEQ ID NO: 31.

In some embodiments, the light chain comprises a functional variant of the amino acid sequence as set forth in SEQ ID NO: 15 or SEQ ID NO: 30 formed by insertion, deletion and/or substitution of one or more amino acid(s) therein, provided that the functional variant retains the ability of binding to GPC3 and CD3. In some embodiments, the heavy chain comprises a functional variant of the amino acid sequence as set forth in SEQ ID NO: 20 or SEQ ID NO: 31 formed by insertion, deletion and/or substitution of one or more amino acid(s) therein, provided that the functional variant retains the ability of binding to GPC3 and CD3.

The functional variants of SEQ ID NOs: 15, 20, 30, and 31 can be those as described above.

In a preferred embodiment, the light chain comprises an amino acid sequence as set forth in SEQ ID NO: 15 and the heavy chain comprises an amino acid sequence as set forth in SEQ ID NO: 20; or the light chain comprises an amino acid sequence as set forth in SEQ ID NO: 30 and the heavy chain comprises an amino acid sequence as set forth in SEQ ID NO: 31.

In some embodiments, the bispecific antibody can be a bispecific T-cell engager (BiTE), preferably an HBiTE as described above.

In still another aspect, the present disclosure provides a nucleic acid comprising a nucleotide sequence encoding the antibody or the antigen binding fragment thereof disclosed herein or the bispecific antibody or the antigen binding fragment thereof disclosed herein.

In yet another aspect, the present disclosure provides a vector comprising the nucleic acid disclosed herein.

Any vector may be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pGAR, pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

A recombinant expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also may be used. Examples of plant expression vectors useful in the context of the disclosure comprise pBI01, pBI101.2, pBI101.3, pBI121 and pBIN19 (Clontech). Examples of animal expression vectors useful in the context of the disclosure comprise pcDNA, pEUK-Cl, pMAM, and pMAMneo (Clontech).

Recombinant expression vectors may be prepared using standard recombinant DNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, may be prepared to contain a replication system functional in a prokaryotic or eukaryotic host cell. Replication systems may be derived, e.g., from ColE1, 2μ plasmid, λ, SV40, bovine papilloma virus, and the like.

In another aspect, the present disclosure provides a host cell comprising the nucleic acid disclosed herein or the vector disclosed herein.

Any cell may be used as a host cell for the nucleic acids or the vectors of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some embodiments, the cell is a human cell. In some embodiments, the cell is an immune cell. In some embodiments, host cells include, for example, CHO cells, such as CHOS cells and CHO-K1 cells, or HEK293 cells, such as HEK293A, HEK293T and HEK293FS.

In still another aspect, the present disclosure provides a pharmaceutical composition comprising (i) the antibody or the antigen binding fragment thereof disclosed herein, or the bispecific antibody or the antigen binding fragment thereof disclosed herein; and (ii) a pharmaceutically acceptable carrier or excipient.

In some embodiments, the carrier or excipient for use with the composition disclosed herein includes but is not limited to maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments of the pharmaceutical composition disclosed herein, the pharmaceutical composition further comprises a second therapeutic agent. In some embodiments, the second therapeutic agent can be selected from an antibody, a chemotherapeutic agent and a small molecule drug. In some embodiments, the second therapeutic agent can be selected from a Bruton's tyrosine kinase (BTK) inhibitor, a PI3K inhibitor, a HDAC inhibitor, a PD-1/PD-L1 inhibitor, a LAG3 inhibitor, and glucocorticoid, or any combination thereof.

In some embodiments, the therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In yet another aspect, the present disclosure provides a conjugate comprising the antibody or the antigen binding fragment thereof disclosed herein or the bispecific antibody or the antigen binding fragment thereof disclosed herein, and a chemical moiety conjugated thereto.

In some embodiments of the conjugate disclosed herein, the chemical moiety is selected from the group consisting of a therapeutic agent, a detectable moiety, and an immunostimulatory molecule.

In some embodiments, the therapeutic agent includes but is not limited to immunomodulators, radioactive compounds, enzymes (for example perforin), chemotherapeutic agents (for example cis-platin), or a toxin. In some embodiments, the therapeutic agent can be such as maytansine, geldanamycin, tubulin inhibitors such as tubulin binding agents (e.g., auristatins), or minor groove binding agents such as calicheamicin.

Other suitable therapeutic agents include such as, small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin: peptide cytotoxins, i.e. proteins or fragments thereof with the ability to kill mammalian cells, for example, ricin, diphtheria toxin, pseudomonas bacterial exotoxin A, DNase and RNase: radio-nuclides, i.e. unstable isotopes of elements which decay with the concurrent emission of one or more of α or β particles, or γ rays, for example, iodine-131, rhenium-186, indium-111, yttrium-90, bismuth-210, bismuth-213, actinium-225 and astatine-213: chelating agents may be used to facilitate the association of these radionuclides to the molecules, or multimers thereof.

In some embodiments, the detectable moiety can be selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. A detectable moiety for diagnostic purposes include for instance, fluorescent labels, radiolabels, enzymes, nucleic acid probes and contrast reagents.

In some embodiments, the immunostimulatory molecule is an immune effector molecules which stimulate immune response. For example, the immunostimulatory molecule can be cytokines such as IL-2 and IFN-γ, chemokines such as IL-8, platelet factor 4, melanoma growth stimulatory protein, complement activators: viral/bacterial protein domains, or viral/bacterial peptides.

In another aspect, the present disclosure provides a method of treating a cancer in a subject comprising administering to the subject an effective amount of the antibody or the antigen binding fragment thereof disclosed herein, the bispecific antibody or the antigen binding fragment thereof disclosed herein, the pharmaceutical composition disclosed herein, or the conjugate disclosed herein.

In some embodiments of the method disclosed herein, the cancer is a GPC3 positive cancer. In some embodiments, the cancer is selected from the group consisting of liver cancer, colon cancer, pancreatic cancer, breast cancer, lung cancer, ovarian cancer, esophageal cancer, bladder cancer, prostate cancer, colorectal cancer, uterine cancer, cervical cancer, brain cancer, cervical cancer, gastric cancer, cholangiocarcinoma, chondrosarcoma, kidney cancer, thyroid cancer, skin cancer, melanoma, glioma, neuroblastoma, lymphoma and myeloma. Preferably, the cancer is selected from the group consisting of selected from the group consisting of liver cancer, colon cancer (such as colon adenocarcinoma and colorectal carcinoma), pancreatic cancer, lung cancer (such as lung mesothelioma, non-small cell lung cancer (NSCLC) and lung squamous cell carcinoma), bladder cancer, melanoma and myeloma (such as multiple myeloma).

In some embodiments, dosage administered to a subject may vary with the embodiment, the medicament employed, the method of administration, and the site and subject being treated. However, a dose should be sufficient to provide a therapeutic response. A clinician may determine the effective amount to be administered to a human or other subject in order to treat a medical condition. The precise amount required to be therapeutically effective may depend upon numerous factors, e.g., such as the activity of the antibody, and the route of administration.

A dose of the antibodies, compositions or conjugates described herein may be administered to a mammal at one time or in a series of sub-doses administered over a suitable period of time, e.g., on a daily, semi-weekly, weekly, bi-weekly, semi-monthly, bi-monthly, semi-annual, or annual basis, as needed. A dosage unit comprising an effective amount of antibodies, compositions or conjugates may be administered in a single daily dose, or the total daily dosage may be administered in two, three, four, or more divided doses administered daily, as needed.

A suitable means of administration may be selected by a medical practitioner. Route of administration may be parenteral, for example, administration by injection, transnasal administration, transpulmonary administration, or transcutaneous administration. Administration may be systemic or local by intravenous injection, intramuscular injection, intraperitoneal injection, subcutaneous injection. In some embodiments, the antibodies, compositions or conjugates are selected for parenteral delivery, for inhalation, or for delivery through the digestive tract, such as orally. Dose and method of administration may vary depending on the weight, age, condition, and the like of the subject, and may be suitably selected.

In some embodiments, the method further comprises administering to the subject a second therapeutic agent. In certain embodiments, a binding agent is administered prior to, substantially simultaneously with, or after the administration of the second therapeutic agent.

In some embodiments, the second therapeutic agent is selected from an antibody, a chemotherapeutic agent and a small molecule drug. In some preferred embodiments, the second therapeutic agent can be selected from a Bruton's tyrosine kinase (BTK) inhibitor, a PI3K inhibitor, a HDAC inhibitor, a PD-1/PD-L1 inhibitor, a LAG3 inhibitor, and glucocorticoid, or any combination thereof.

In some embodiments, the second therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In still another aspect, the present disclosure provides a method of detecting GPC3 positive cancer in a subject comprising (i) contacting a sample obtained from the subject with the antibody or the antigen binding fragment thereof disclosed herein, the bispecific antibody or the antigen binding fragment thereof disclosed herein, or the conjugate disclosed herein; and (ii) detecting binding of the antibody or the antigen binding fragment thereof to GPC3 in the sample.

In some embodiments, the antibody or the antigen binding fragment thereof is linked to a detectable moiety. The detectable moiety can be selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. A detectable moiety for diagnostic purposes include for instance, fluorescent labels, radiolabels, enzymes, nucleic acid probes and contrast reagents.

In some embodiments, the cancer is selected from the group consisting of liver cancer, colon cancer, pancreatic cancer, breast cancer, lung cancer, ovarian cancer, esophageal cancer, bladder cancer, prostate cancer, colorectal cancer, uterine cancer, cervical cancer, brain cancer, cervical cancer, gastric cancer, cholangiocarcinoma, chondrosarcoma, kidney cancer, thyroid cancer, skin cancer, melanoma, glioma, neuroblastoma, lymphoma and myeloma. Preferably, the cancer is selected from the group consisting of selected from the group consisting of liver cancer, colon cancer (such as colon adenocarcinoma and colorectal carcinoma), pancreatic cancer, lung cancer (such as lung mesothelioma, non-small cell lung cancer (NSCLC) and lung squamous cell carcinoma), bladder cancer, melanoma and myeloma (such as multiple myeloma).

In yet another aspect, the present disclosure provides a kit for detecting the presence of a GPC3 antigen in a sample comprising the antibody or the antigen binding fragment thereof disclosed herein, the bispecific antibody or the antigen binding fragment thereof disclosed herein, or the conjugate disclosed herein. Preferably, the antibody or the antigen binding fragment thereof is linked to a detectable moiety. The detectable moiety can be selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. A detectable moiety for diagnostic purposes include for instance, fluorescent labels, radiolabels, enzymes, nucleic acid probes and contrast reagents.

In another aspect, the present disclosure provides use of the antibody or the antigen binding fragment thereof disclosed herein, the bispecific antibody or the antigen binding fragment thereof disclosed herein, the pharmaceutical composition disclosed herein, or the conjugate disclosed herein in the manufacture of a medicament for treating a cancer in a subject. In some embodiments, the cancer is a GPC3 positive cancer.

In still another aspect, the present disclosure provides the antibody or the antigen binding fragment thereof disclosed herein, the bispecific antibody or the antigen binding fragment thereof disclosed herein, the pharmaceutical composition disclosed herein, or the conjugate disclosed herein for use in treating a cancer in a subject. In some embodiments, the cancer is a GPC3 positive cancer.

In yet another aspect, the present disclosure provides use of the antibody or the antigen binding fragment thereof disclosed herein, the bispecific antibody or the antigen binding fragment thereof disclosed herein, or the conjugate disclosed herein in the manufacture of a kit for detecting GPC3 positive cancer in a subject.

In still another aspect, the present disclosure provides the antibody or the antigen binding fragment thereof disclosed herein, the bispecific antibody or the antigen binding fragment thereof disclosed herein, or the conjugate disclosed herein for use in detecting GPC3 positive cancer in a subject.

In some embodiments of the use disclosed herein, the GPC3 positive cancer is selected from the group consisting of liver cancer, colon cancer, pancreatic cancer, breast cancer, lung cancer, ovarian cancer, esophageal cancer, bladder cancer, prostate cancer, colorectal cancer, uterine cancer, cervical cancer, brain cancer, cervical cancer, gastric cancer, cholangiocarcinoma, chondrosarcoma, kidney cancer, thyroid cancer, skin cancer, melanoma, glioma, neuroblastoma, lymphoma and myeloma. Preferably, the cancer is selected from the group consisting of selected from the group consisting of liver cancer, colon cancer (such as colon adenocarcinoma and colorectal carcinoma), pancreatic cancer, lung cancer (such as lung mesothelioma, non-small cell lung cancer (NSCLC) and lung squamous cell carcinoma), bladder cancer, melanoma and myeloma (such as multiple myeloma).

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

Cell lines including Hep-G2 (human liver cancer cell line), A375 (human melanoma cell line), HuH7 (hepatocyte derived cellular carcinoma cell line), SK-HEP-1 (human hepatic adenocarcinoma cell line), A549 (human non-small cell lung cancer cell line), LS174T (human colon adenocarcinoma cell line) RPMI8226 (human myeloma cell line), H226 (human lung mesothelioma cell line), and 5637 (human bladder carcinoma cell line) were purchased from National Collection of Authenticated Cell Cultures.

A tumor cell line stably expressing GPC3, LS174T-GPC3, was generated by transfection of the commercial GPC3 recombinant plasmid pCMV-GPC3 (Sino Biological) into LS174T cells using the agent Lipofectamine™ LTX Reagent with PLUS™ Reagent (Thermo) and the stable cell line LS174T-GPC3 was obtained by hygromycin B screening.

Biotinylated human GPC3 protein, human GPC3 protein, cynomolgus GPC3 protein, and mouse GPC3 protein were purchased from ACROBiosystems. Anti-human IgG (γ-chain specific)-R-PE antibody, anti-human IgG (Fc-specific)-peroxidase antibody, and monoclonal anti-Flag® RM2-peroxidase were purchased from Sigma.

M13KO7 helper phage was purchased from New England Biolabs. Dynabeads™ Myone™ Streptavidin T1 was purchased from ThermoFisher Scientific. PE anti-His tag antibody was purchased from BioLegend. M13 bacteriophage antibody (HRP) was purchased from Sino Biological.

### Example 1. Panning and Screening of a Phage-Display Naive Human Fab Library for Identification of GPC3 Antibodies

A large (size, 10¹¹) phage-display naive human Fab library with peripheral blood B cells from about 30 healthy individuals was used for selection of antibodies against recombinant human GPC3 conjugated to magnetic beads (Dynabeads™ Myone™ Streptavidin T1: ThermoFisher Scientific) as described previously (Zhu et al., J Virol 2006, 80:891-899) with minor modification that 5, 1 and 0.2 mg of antigen was used in the first, second and third round of panning, respectively. Strong positive signals were observed from the 3th round of biopanning by using polyclonal phage ELISA. The 3th round phage was subsequently subjected to test for its specific binding. By soluble expression-based monoclonal enzyme-linked immunosorbent assay (SemELISA) and sequencing analysis, two specific Fab clones, designated as 1A1 and 6A4, were identified. Both 1A1 and 6A4 Fabs have a k light chain.

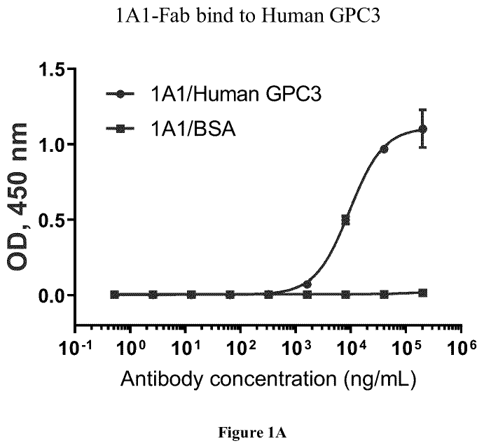

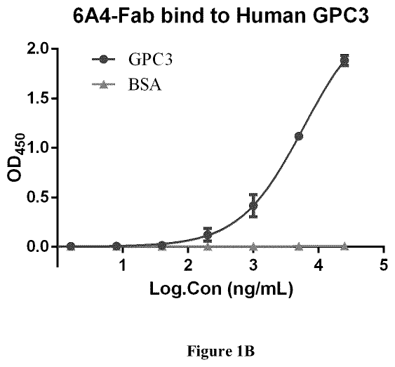

The hexahistidine-tagged 1A1 Fab and 6A4 Fab were expressed in E. coli strain HB2151 and purified from the soluble fraction of periplasm by using the Ni-NTA resin. Then ELISA was performed by using standard protocols to measure binding affinity to recombinant human GPC3 (full-length extracellular domain). Briefly, the recombinant human GPC3 (ACROBiosystems) was coated on Corning EIA/RIA high-binding 96-well plates (Corning Inc.) at 50 ng per well overnight at 4° C. and blocked with 3% nonfat milk in PBS (pH7.4). Fivefold serially diluted antibodies were added and incubated at room temperature for 2 h. The plates were washed with PBS containing 0.05% Tween 20. Bound antibodies were detected by HRP-conjugated anti-FLAG tag antibody (Sino Biological). The assay was developed at room temperature with TMB substrate (Solarbio) and OD value was measured at 450 nm with a microplate reader. The results showed that Fab clone 1A1 has an affinity with EC₅₀ of approximately 190 nM (FIG. 1A), and Fab clone 6A4 has an affinity with EC₅₀ of approximately 234 nM (FIG. 1B).

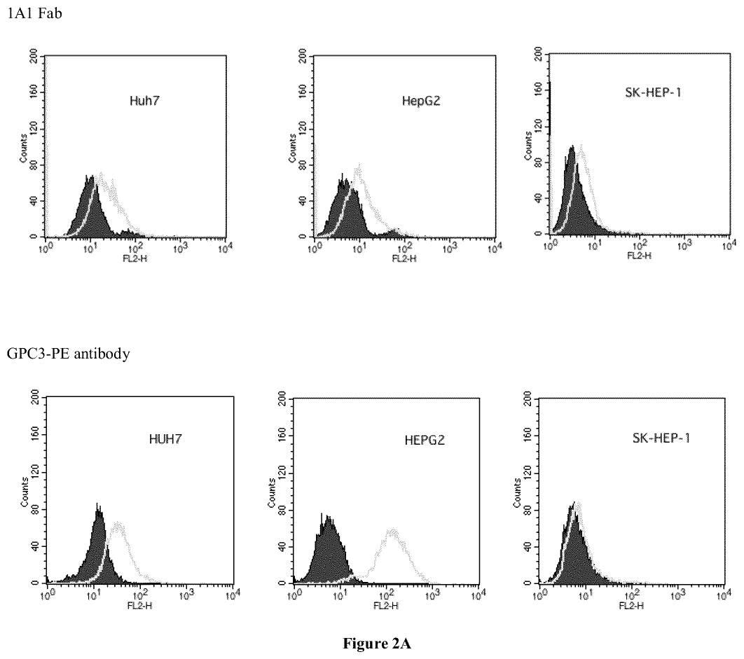

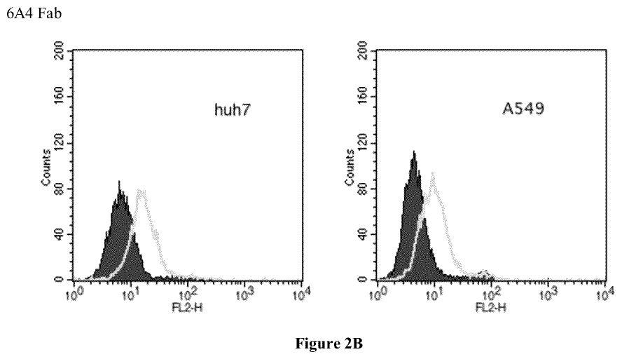

To measure the binding of 1A1 Fab or 6A4 Fab against cell surface-associated GPC3, flow cytometry was carried out with cancer cell lines HepG2, HuH7, SK-HEP-1 and A549. 5×10⁵ cells of each cell line was incubated with Fab antibodies (10 μg/ml) or GPC3-PE antibody (Sino biological, 10 μg/ml) as a positive control on ice for 60 min. The cells were washed once with PBS containing 0.1% bovine serum albumin (PBSA) and resuspended in 200 ml PBSA. Then 2 μl anti-His-PE conjugate (BioLegend) was added and incubated for 60 min. The cells were washed once with PBSA and then used for flow cytometry analysis. The results are shown in FIGS. 2A and 2B.

As can be seen from FIG. 2A, 1A1 Fab shows moderate binding to HepG2 and HuH7, and relatively weak binding to SK-HEP-1. This may be due to the relative low expression of GPC3 on SK-HEP-1 cells, as evidenced by the nearly no binding of GPC3-PE antibody to these cells. As can be seen from FIG. 2B, 6A4 Fab shows moderate binding to HuH7 and A549. The results suggest that 1A1 Fab and 6A4 Fab can bind well to cancer cell lines expressing GPC3.

### Example 2. Construction and Initial Characterization of Anti-GPC3 Monoclonal Antibodies

Fab clones 1A1 and 6A4 was used to construct intact monoclonal antibodies (1A1 mAb and 6A4 mAb). Briefly, the heavy chain Fd fragments of Fab clones 1A1 and 6A4 were fused to the N-terminus of human IgG1 Fc fragment, respectively. Both light chain and heavy chain were constructed into the vector pDin1, which was modified from pDR12 by the inventors to comprise two molecular cloning sites (MCS) for the expression of monoclonal antibodies. Construction and initial characterization of the anti-GPC3 1A1 mAb and 6A4 mAb were performed as follow.

### Cloning of Anti-GPC3 Monoclonal Antibodies

To generate the construct of anti-GPC3 1A1 mAb. following primers were used:

GPC3-1A1-IgG1-VH-FP-HindIII,
(SEQ ID NO: 33)
5′ GAATAAGCTTGCCGCCACCATGGAATGGAGC
TGGGTCTTTCTCTTCTTCCT′ 3′ (sense);
GPC3-IgG1-FC-RP-Xba I ,
(SEQ ID NO: 34)
5′ GTACTCTAGATTATTTACCCGGAGACAGGGAGAGG
CTCTTCTGCGTGTAGTGGTTG 3′ (antisense);
GPC3-1A1-IgG1-VL-FP-NotI,
(SEQ ID NO: 35)
5′ AGTCCGCGGCCGCGCCACCATGGGTGTGCCCACT
CAGGTCCTGGGGT 3′ (sense);
GPC3-1A1-IgG1-LC-RP-XhoI,
(SEQ ID NO: 36)
5′ GCATCTCGAGTTAACACTCTCCCCTGTTGAAGCT
CTTT 3′ (antisense);
GPC3-1A1-IgG1-VH-RP-OL,
(SEQ ID NO: 37)
5′ TGTGTGAGTTTTGTCACAAGATTTGGGCTCAACTT
TCTT 3′ (sense);
GPC3-IgG1-FC-FP-OL,
(SEQ ID NO: 38)
5′ TGTGACAAAACTCACACATGTCCACCGTGCCCAGCA 3′
(antisense).

For the generation of anti-GPC3 1A1 mAb, the gene fragments of VL+CL and VH+CH1 of anti-GPC3 antibody were amplified from anti-GPC3 1A1 Fab with primer pairs GPC3-1A1-IgG1-VL-FP-NotI/GPC3-1A1-IgG1-LC-RP-XhoI and GPC3-1A1-IgG1-VH-FP-HindIII/GPC3-1A1-IgG1-VH-RP-OL, respectively. The Fc domain was amplified from pDin1 vector containing a monomeric Fc fragment of IgG1 with primer pairs GPC3-IgG1-FC-FP-OL/GPC3-IgG1-FC-RP-Xba I. For the full-length heavy chain, the PCR products were fused to Fc domain by overlapping PCR using the primer pairs GPC3-1A1-IgG1-VH-FP-HindIII/GPC3-IgG1-FC-RP-Xba I. The heavy chain gene fragment was digested with HindIII and XbaI and cloned into pBudCE4.1 vector. The light chain gene fragment was cloned into pBudCE4.1 vector via the NotI and XhoI restriction sites. These two vectors were used together for expression of anti-GPC3 1A1 mAb.

To generate the construct of anti-GPC3 6A4 mAb, following primers were used:

GPC3-6A4-Mab-VH-FP-OL,
(SEQ ID NO: 39)
5′ CAGCACTGCTCTGTTGCCTGGTCCTCCTGACTGGG
GTGAGGGCCGAAGTGCAGCTGGTG 3′ (sense);
GPC3-6A4-Mab-VH-RP-OL,
(SEQ ID NO: 40)
5′ GGCATGTGTGAGTTTTGTCACAAGATTTGGGCTCA
ACTTTCTTGT 3′ (antisense);
GPC3-6A4-Mab-Fc-FP-OL,
(SEQ ID NO: 41)
5′ GTGACAAAACTCACACATGCC 3′ (sense);
GPC3-6A4-Mab-Fc-RP-Xbal,
(SEQ ID NO: 42)
5′ CGATTCTAGAATCATTTACCCGGGGACAGGGAG
AGGCT 3′ (antisense);
GPC3-6A4-Mab-VL-FP-OL,
(SEQ ID NO: 43)
5′ GCACTGCTCTGTTGCCTGGTCCTCCTGACTGGGGT
GAGGGCCGATGTTGTGATGACT 3′ (sense);
GPC3-6A4-Mab-VL-RP-Xbal,
(SEQ ID NO: 44)
5′ CGATTCTAGAATCAACACTCTCCCCTGTTGAAGC
TCTT 3′ (antisense);
pBY-SP-FP-Not1,
(SEQ ID NO: 45)
5′ GAATGCGGCCGCAAACTACAAGACAGACTTGCAAAAGAAG
GCATGCACAGCTCAGCACTGCTCTGTTG 3′ (sense).

For the generation of anti-GPC3 6A4 mAb, the gene fragments of light chain and heavy chain of anti-GPC3 6A4 mAb were obtained by using a similar protocol to 1A1 mAb. The gene fragments were cloned into pBY vector via the NotI and XbaI restriction sites.

### Protein Expression, Purification and Initial Characterization

Anti-GPC3 1A1 mAb and 6A4 mAb were expressed in either 293FS or CHO-S cells. The plasmids and transfection agent PEI were mixed at ratio 1:3 and then dropwise added into 293FS or CHO-S cell culture. The cells were continued to grow for 5-7 days after transfection. The cell culture was harvested by centrifugation at 8000 rpm for 20 min. The culture supernatant containing target proteins were loaded onto Protein A Sepharose 4 Fast Flow column chromatography (GE Healthcare), and purified according to the manufacturer's instructions.

The purified proteins were subjected to SDS-PAGE. On a non-reducing SDS-PAGE, 1A1 mAb displays an apparent molecular weight (aMW) of approximately 150 kDa. On a reducing SDS-PAGE, the heavy chain and light chain have apparent molecular weight of approximately 55 kDa and 30 kDa, respectively (data not shown). The CDR sequences of 1A1 mAb and 6A4 mAb according to the Kabat numbering system are shown in Table 1. The amino acid sequences of light chain variable region (VL) and heavy chain variable region (VH) are shown in Table 2. The whole light chain and heavy chain sequences of 1A1 mAb and 6A4 mAb are shown in Table 3.

TABLE 1
CDR sequences of 1A1 mAb and 6A4 mAb

| | | |
|---|---|---|
| 1A1 mAb | LCDR1 | RSSQSLVYSDGNTYLN (SEQ ID NO: 1) |
| | LCDR2 | KVSNRDS (SEQ ID NO: 2) |
| | LCDR3 | MQSTHWPLT (SEQ ID NO: 3) |
| | HCDR1 | SYGIS (SEQ ID NO: 6) |
| | HCDR2 | WISAYNGNTNYAQKLQG (SEQ ID NO: 7) |
| | HCDR3 | AGTPTQILRYFDWLSQPFDY (SEQ ID NO: 8) |
| 6A4 mAb | LCDR1 | RASQSISSYLN (SEQ ID NO: 23) |
| | LCDR2 | AASSLQS (SEQ ID NO: 24) |
| | LCDR3 | QQSYSTPLT (SEQ ID NO: 25) |
| | HCDR1 | SYAMH (SEQ ID NO: 60) |
| | HCDR2 | WINAGNGNTKYSQKFQG (SEQ ID NO: 61) |
| | HCDR3 | DPSH (SEQ ID NO: 62) |

TABLE 2
VL and VH sequences of 1A1 mAb and 6A4 mAb

| | | |
|---|---|---|
| 1A1 mAb VL | EIVLTQSPLSLPVTLGQPASISCRSSQSLVYSDGNTYL | SEQ ID NO: 4 |
| | NWFHQRPGQSPRRLIYKVSNRDSGVPERFSGSGSGT | |
| | DFTLKISRVEAEDVGVYYCMQSTHWPLTFGGGTKV | |
| | EIK | |
| 1A1 mAb VH | EVQLVQSGAEVKKPGASVKVSCKASGYTFTSYGISW | SEQ ID NO: 9 |
| | VRQAPGQGLEWMGWISAYNGNTNYAQKLQGRVTM | |
| | TTDTSTSTAYMELRSLRSDDTAVYYCARAGTPTQIL | |
| | RYFDWLSQPFDYWGQGTLVTVSS | |
| 6A4 mAb VL | DVVMTQSPSSLSASVGDRVTITCRASQSISSYLNWYQ | SEQ ID NO: 26 |
| | QKPGKAPKLLIYAASSLQSGVPSRFSGSGSGTDFTLTI | |
| | SSLQPEDFATYYCQQSYSTPLTFGGGTKVEIK | |
| 6A4 mAb VH | EVQLVQSGAEVKKPGASVKVSCKASGYTFTSYAMH | SEQ ID NO: 28 |
| | WVRQAPGQRLEWMGWINAGNGNTKYSQKFQGRVT | |
| | ITRDTSTSTAYMELRSLRSDDTAVYYCARDPSHWGQ | |
| | GTLVTVSS | |

TABLE 3
Light chain and heavy chain sequences of 1A1 mAb and 6A4 mAb

| | | |
|---|---|---|
| 1A1 mAb | EIVLTQSPLSLPVTLGQPASISCRSSQSLVYSDGNTYLN | SEQ ID NO: 5 |
| light chain | WFHQRPGQSPRRLIYKVSNRDSGVPERFSGSGSGTDF | |
| | TLKISRVEAEDVGVYYCMQSTHWPLTFGGGTKVEIK | |
| | RTVAAPSVFIFPPSDEQLKSGTASVVCLLNNFYPREAK | |
| | VQWKVDNALQSGNSQESVTEQDSKDSTYSLSSTLTLS | |
| | KADYEKHKVYACEVTHQGLSSPVTKSFNRGEC | |
| 1A1 mAb | EVQLVQSGAEVKKPGASVKVSCKASGYTFTSYGISW | SEQ ID NO: 10 |
| heavy chain | VRQAPGQGLEWMGWISAYNGNTNYAQKLQGRVTM | |
| | TTDTSTSTAYMELRSLRSDDTAVYYCARAGTPTQILR | |
| | YFDWLSQPFDYWGQGTLVTVSSASTKGPSVFPLAPSS | |
| | KSTSGGTAALGCLVKDYFPEPVTVSWNSGALTSGVH | |
| | TFPAVLQSSGLYSLSSVVTVPSSSLGTQTYICNVNHKP | |
| | SNTKVDKKVEPKSCDKTHTCPPCPAPELLGGPSVFLFP | |
| | PKPKDTLMISRTPEVTCVVVDVSHEDPEVKFNWYVD | |
| | GVEVHNAKTKPREEQYNSTYRVVSVLTVLHQDWLN | |
| | GKEYKCKVSNKALPAPIEKTISKAKGQPREPQVYTLPP | |
| | SRDELTKNQVSLTCLVKGFYPSDIAVEWESNGQPENN | |
| | YKTTPPVLDSDGSFFLYSKLTVDKSRWQQGNVFSCSV | |
| | MHEALHNHYTQKSLSLSPGK | |
| 6A4 mAb | DVVMTQSPSSLSASVGDRVTITCRASQSISSYLNWYQ | SEQ ID NO: 27 |
| light chain | QKPGKAPKLLIYAASSLQSGVPSRFSGSGSGTDFTLTIS | |
| | SLQPEDFATYYCQQSYSTPLTFGGGTKVEIKRTVAAPS | |
| | VFIFPPSDEQLKSGTASVVCLLNNFYPREAKVQWKVD | |
| | NALQSGNSQESVTEQDSKDSTYSLSSTLTLSKADYEK | |
| | HKVYACEVTHQGLSSPVTKSFNRGEC | |
| 6A4 mAb | EVQLVQSGAEVKKPGASVKVSCKASGYTFTSYAMH | SEQ ID NO: 29 |
| heavy chain | WVRQAPGQRLEWMGWINAGNGNTKYSQKFQGRVTI | |
| | TRDTSTSTAYMELRSLRSDDTAVYYCARDPSHWGQG | |
| | TLVTVSSASTKGPSVFPLAPSSKSTSGGTAALGCLVKD | |
| | YFPEPVTVSWNSGALTSGVHTFPAVLQSSGLYSLSSV | |
| | VTVPSSSLGTQTYICNVNHKPSNTKVDKKVEPKSCDK | |
| | THTCPPCPAPELLGGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKALPAPI | |
| | EKTISKAKGQPREPQVYTLPPSRDELTKNQVSLTCLV | |
| | KGFYPSDIAVEWESNGQPENNYKTTPPVLDSDGSFFL | |
| | YSKLTVDKSRWQQGNVFSCSVMHEALHNHYTQKSLS | |
| | LSPGK | |

### Example 3. Construction and Initial Characterization of Anti-GPC3 Bispecific Antibody

Bispecific T cell engager (BiTE) is a novel class of bispecific antibodies which guide cytotoxic T cells to kill cancer cells by simultaneously binding to a tumor antigen and a T cell antigen, such as CD3 molecule on T cell surface. HBiTE as described in PCT application No. PCT/US2018/016524 (which is incorporated herein by reference in its entirety) is a specific form of BiTE. HBiTE has a light chain and a heavy chain forming a heterodimer. The light chain, from N-terminus to C-terminus, comprises an anti-target (e.g. tumor antigen) VL domain, an anti-CD3 VL-CL and a monomeric human IgG1 Fc (e.g., mFc7.2). The heavy chain, from N-terminus to C-terminus, comprises an anti-target VH domain, an anti-CD3 VH-CH1 and a monomeric human IgG1 Fc (e.g., mFc7.2). Monomeric Fc7.2 contains two amino acid mutations (T366L and Y407H) capable of inhibiting Fc homodimerization. To generate GPC3×CD3 HBiTE, the VL and VH domains of the above anti-GPC3 antibody were fused to the N-terminus of VL and VH domains of anti-CD3 Fab via linkers GGGGSGGGGSGGGGS (SEQ ID NO: 21) or GSGGGGSGGGGS (SEQ ID NO: 32) and GGGSSGGGGSGGGGS (SEQ ID NO: 22), respectively. The anti-CD3 Fab is further fused to the N terminus of mFc7.2. The light chain and heavy chain were constructed into the vector pDin1 for expression in mammalian cells. Construction and initial characterization of the bispecific antibodies targeting GPC3 and CD3 (1A1-based GPC3×CD3 HBiTE and 6A4-based GPC3×CD3 HBITE) were performed as follow.

### Cloning of the Bispecific Antibody Targeting GPC3 and CD3

To generate constructs of the bispecific antibody 1A1-based GPC3×CD3 HBITE, following primers were used:

bnIgG20L1,
(SEQ ID NO: 46)
5′ GTGTAAGCTTACCATGGGTGTGCCCACTCAGGTCCTGGGGT 3′
(sense);
BI-GPC3-VL-FP,
(SEQ ID NO: 47)
5′ CAGGTGTCCACTCCGAAATTGTGCTGACTCAG 3′ (sense);
BI-GPC3-VL-RP,
(SEQ ID NO: 48)
5′ AGGGGGATCCTTTGATCTCCACCTTGGTCCCTCCGCCGAAA
GT 3′ (antisense);
bnIgG20H1,
(SEQ ID NO: 49)
5′ GTGTTCTAGAGCCGCCACCATGGAATGGAGCTGGGTCTTTC 3′
(sense);
BI-GPC3-VH-FP,
(SEQ ID NO: 50)
5′ GGCTTACAGATGCCAGATGTGAGGTGCAGCTGGTGCAG 3′
(sense);
GPC3HB-VH-RP-correct,
(SEQ ID NO: 51)
5′ GATAGAGCTCGAGGAGACGGTGACCAGGGTT 3′
(antisense).

For the generation of 1A1-based GPC3×CD3 HBITE, the gene fragments of VL and VH domains were amplified from anti-GPC3 1A1 Fab with primer pairs BI-GPC3-VL-FP/BI-GPC3-VL-RP and BI-GPC3-VH-FP/GPC3HB-VH-RP-correct, respectively. The PCR products were fused to the 3′ end of H leader and L leader by overlapping PCR using the primer pairs bnIgG20H1/BI-GPC3-VL-RP and bnIgG20L1/GPC3HB-VH-RP-correct, respectively. The H leader-VL gene fragment was digested with XbaI and BamHI and cloned into HBiTE derived pDin1 vector containing an anti-CD3 hSP34 Fab and a complete Fc fragment. The L leader-VH gene fragment was then further cloned into the recombinant plasmid containing the H leader-VL insert via the HindIII and SacI restriction sites.

To generate constructs of the bispecific antibody 6A4-based GPC3×CD3 HBITE, following primers were used:

BI-011-6A4-VL-FP,
(SEQ ID NO: 52)
5′ TCAGCACTGCTCTGTTGCCTGGTCCTCCTGACTGGGGTGA
GGGCCGATGTTGTGATGACTCAGT 3′ (sense);
BI-011-6A4-VL-RP,
(SEQ ID NO: 53)
5′ GCCAGAGCCACCTCCGCCGGATCCTTTGATCTCCACCTTG
GTCCCT 3′ (antisense);
pBY-SP-FP-Not I,
(SEQ ID NO: 54)
5′ GCGGCCGCAAACTACAAGACAGACTTGCAAAAGAAGGCA
TGCACAGCTCAGCACTGCTCTGT 3′ (sense);
CD3-VL-FP,
(SEQ ID NO: 55)
5′ GGATCCGGCGGAGGTGGCTCTGGC 3′ (sense);
FC-RP-Xba I,
(SEQ ID NO: 56)
5′ TGATCTAGAATTATTTACCCGGAGACAGGGAGAGGCTCT 3′
(antisense);
BI-011-6A4-VH-FP,
(SEQ ID NO: 57)
5′ GCTCAGCACTGCTCTGTTGCCTGGTCCTCCTGACTGGGGT
GAGGGCCGAAGTGCAGCTGGTGCA 3′ (sense);
BI-011-6A4-VH-RP,
(SEQ ID NO: 58)
5′ ACCTCCGCCTGAGCTCCCTCCACCTGAGGAGACGGTGAC
CAGGGT 3′ (antisense);
CD3-VH-FP,
(SEQ ID NO: 59)
5′ GGTGGAGGGAGCTCAGGCGGAGGT 3′ (sense).

For the generation of 6A4-based GPC3×CD3 HBITE, a plasmid pWCI-GPC3-6A4 was amplified using primer pairs BI-011-6A4-VL-FP and BI-011-6A4-VL-RP to obtain the gene fragment of VL. The gene fragment of VL was then amplified using primer pairs pBY-SP-FP-Not I and BI-011-6A4-VL-RP to obtain the gene fragment of SP+VL. A plasmid pDin1-GPC3-1A1 was amplified using primer pairs CD3-VL-FP and FC-RP-Xba I to obtain the gene fragment of FC. The gene fragments of SP+VL and FC were amplified using primer pairs pBY-SP-FP-Not I and FC-RP-Xba I to obtain complete gene fragment of light chain. The light chain gene fragment was digested with Not I and XbaI and cloned into pBY vector.

A plasmid pWCI-GPC3-6A4 was amplified using primer pairs BI-011-6A4-VH-FP and BI-011-6A4-VH-RP to obtain the gene fragment of VH. The gene fragment of VH was then amplified using primer pairs pBY-SP-FP-Not I and BI-011-6A4-VH-RP to obtain the gene fragment of SP+VH. A plasmid pDin1-GPC3-1A1 was amplified using primer pairs CD3-VH-FP and FC-RP-Xba I to obtain the gene fragment of FC. The gene fragments of SP+VH and FC were amplified using primer pairs pBY-SP-FP-Not I and FC-RP-Xba I to obtain complete gene fragment of heavy chain. The heavy chain gene fragment was digested with Not I and XbaI and cloned into pBY vector.

### Protein Expression, Purification and Initial Characterization

The 1A1-based GPC3×CD3 HBiTE and 6A4-based GPC3×CD3 HBiTE were expressed in either 293FS or CHO-S cells. The plasmids and transfection agent PEI were mixed at ratio 1:3 and then added into 293FS or CHO-S cell culture. The cells were continued to grow for 5-7 days after transfection. The cell culture was harvested by centrifugation at 8000 rpm for 20 min. The culture supernatant containing target proteins were loaded onto Protein A Sepharose 4 Fast Flow column chromatography (GE Healthcare), and purified according to the manufacturer's instructions.

The purified proteins were subjected to SDS-PAGE. On a non-reducing SDS-PAGE, 1A1-based GPC3×CD3 HBiTE displays an apparent molecular weight (aMW) of approximately 120 kDa. On a reducing SDS-PAGE, the heavy chain and light chain are close to each other with an apparent molecular weight of approximately 62 kDa (data not shown). The CDR sequences of 1A1-based GPC3×CD3 HBiTE and 6A4-based GPC3×CD3 HBiTE according to the Kabat numbering system are shown in Table 4. The amino acid sequences of light chain variable region (VL) and heavy chain variable region (VH) are shown in Table 5. The light chain and heavy chain sequences of 1A1-based GPC3×CD3 HBITE and 6A4-based GPC3×CD3 HBiTE are shown in Table 6.

TABLE 4
CDR sequences of 1A1-based GPC3×CD3 HBiTE and 6A4-based GPC3×CD3 HBiTE

| | 1A1-based GPC3×CD3 HBiTE | 6A4-based GPC3×CD3 HBiTE |
|---|---|---|
| LCDR1 against GPC3 | RSSQSLVYSDGNTYLN (SEQ | RASQSISSYLN (SEQ ID NO: |
| | ID NO: 1) | 23) |
| LCDR2 against GPC3 | KVSNRDS (SEQ ID NO: 2) | AASSLQS (SEQ ID NO: 24) |
| LCDR3 against GPC3 | MQSTHWPLT (SEQ ID NO: 3) | QQSYSTPLT (SEQ ID NO: 25) |
| HCDR1 against GPC3 | SYGIS (SEQ ID NO: 6) | SYAMH (SEQ ID NO: 60) |
| HCDR2 against GPC3 | WISAYNGNTNYAQKLQG | WINAGNGNTKYSQKFQG |
| | (SEQ ID NO: 7) | (SEQ ID NO: 61) |
| HCDR3 against GPC3 | AGTPTQILRYFDWLSQPFDY | DPSH (SEQ ID NO: 62) |
| | (SEQ ID NO: 8) | |
| LCDR1 against CD3 | RSSTGAVTTSNYAN (SEQ ID | RSSTGAVTTSNYAN (SEQ ID |
| | NO: 11) | NO: 11) |
| LCDR2 against CD3 | GANKRAP (SEQ ID NO: 12) | GANKRAP (SEQ ID NO: 12) |
| LCDR3 against CD3 | ALWYSNLWV (SEQ ID NO: | ALWYSNLWV (SEQ ID NO: |
| | 13) | 13) |
| HCDR1 against CD3 | GFTFNTY (SEQ ID NO: 16) | GFTFNTY (SEQ ID NO: 16) |
| HCDR2 against CD3 | RSKYNNYA (SEQ ID NO: 17) | RSKYNNYA (SEQ ID NO: 17) |
| HCDR3 against CD3 | HGNFGSSYVSYFAY (SEQ ID | HGNFGSSYVSYFAY (SEQ ID |
| | NO: 18) | NO: 18) |

TABLE 5
VL and VH sequences of 1A1-based GPC3×CD3 HBiTE and 6A4-based GPC3×CD3
HBiTE

| | | | |
|---|---|---|---|
| 1A1-based | VL against | EIVLTQSPLSLPVTLGQPASISCRSSQSLVYSD | SEQ ID NO: |
| GPC3×CD3 | GPC3 | GNTYLNWFHQRPGQSPRRLIYKVSNRDSGVP | 4 |
| HBiTE | | ERFSGSGSGTDFTLKISRVEAEDVGVYYCMQ | |
| | | STHWPLTFGGGTKVEIK | |
| | VL against | EIVVTQSPATLSVSPGERATLSCRSSTGAVTTS | SEQ ID NO: |
| | CD3 | NYANWVQQKPGQAPRGLIGGANKRAPGVPA | 14 |
| | | RFSGSLSGDEATLTISSLQSEDFAVYYCALWY | |
| | | SNLWVFGQGTKLEIK | |
| | VH against | EVQLVQSGAEVKKPGASVKVSCKASGYTFTS | SEQ ID NO: |
| | GPC3 | YGISWVRQAPGQGLEWMGWISAYNGNTNYA | 9 |
| | | QKLQGRVTMTTDTSTSTAYMELRSLRSDDTA | |
| | | VYYCARAGTPTQILRYFDWLSQPFDYWGQG | |
| | | TLVTVSS | |
| | VH against | EVQLVESGGGLVQPGGSLRLSCAASGFTFNT | SEQ ID NO: |
| | CD3 | YAMNWVRQAPGKGLEWVARIRSKYNNYAT | 19 |
| | | YYADSVKGRFTISRDDSKNTLYLQMNSLRAE | |
| | | DTAVYYCARHGNFGSSYVSYFAYWGQGTTV | |
| | | TVSS | |
| 6A4-based | VL against | DVVMTQSPSSLSASVGDRVTITCRASQSISSY | SEQ ID NO: |
| GPC3×CD3 | GPC3 | LNWYQQKPGKAPKLLIYAASSLQSGVPSRFS | 26 |
| HBiTE | | GSGSGTDFTLTISSLQPEDFATYYCQQSYSTPL | |
| | | TFGGGTKVEIK | |
| | VL against | EIVVTQSPATLSVSPGERATLSCRSSTGAVTTS | SEQ ID NO: |
| | CD3 | NYANWVQQKPGQAPRGLIGGANKRAPGVPA | 14 |
| | | RFSGSLSGDEATLTISSLQSEDFAVYYCALWY | |
| | | SNLWVFGQGTKLEIK | |
| | VH against | EVQLVQSGAEVKKPGASVKVSCKASGYTFTS | SEQ ID NO: |
| | GPC3 | YAMHWVRQAPGQRLEWMGWINAGNGNTK | 28 |
| | | YSQKFQGRVTITRDTSTSTAYMELRSLRSDDT | |
| | | AVYYCARDPSHWGQGTLVTVSS | |
| | VH against | EVQLVESGGGLVQPGGSLRLSCAASGFTFNT | SEQ ID NO: |
| | CD3 | YAMNWVRQAPGKGLEWVARIRSKYNNYAT | 19 |
| | | YYADSVKGRFTISRDDSKNTLYLQMNSLRAE | |
| | | DTAVYYCARHGNFGSSYVSYFAYWGQGTTV | |
| | | TVSS | |

TABLE 6
Light chain and heavy chain sequences of 1A1-based GPC3×CD3 HBiTE and
6A4-based GPC3×CD3 HBiTE

| | | |
|---|---|---|
| 1A1-based | EIVLTQSPLSLPVTLGQPASISCRSSQSLVYSDGNTYL | SEQ ID NO: 15 |
| GPC3×CD3 | NWFHQRPGQSPRRLIYKVSNRDSGVPERFSGSGSGT | |
| HBiTE light | DFTLKISRVEAEDVGVYYCMQSTHWPLTFGGGTKV | |
| chain | EIKGGGGSGGGGSGGGGSEIVVTQSPATLSVSPGERA | |
| | TLSCRSSTGAVTTSNYANWVQQKPGQAPRGLIGGA | |
| | NKRAPGVPARFSGSLSGDEATLTISSLQSEDFAVYYC | |
| | ALWYSNLWVFGQGTKLEIKRTVAAPSVFIFPPSDEQ | |
| | LKSGTASVVCLLNNFYPREAKVQWKVDNALQSGNS | |
| | QESVTEQDSKDSTYSLSSTLTLSKADYEKHKVYACE | |
| | VTHQGLSSPVTKSFNRGECPPCPAPELLGGPSVFLFPP | |
| | KPKDTLMISRTPEVTCVVVDVSHEDPEVKFNWYVD | |
| | GVEVHNAKTKPREEQYNSTYRVVSVLTVLHQDWLN | |
| | GKEYKCKVSNKALPAPIEKTISKAKGQPREPQVYTLP | |
| | PSRDELTKNQVSLLCLVKGFYPSDIAVEWESNGQPE | |
| | NNYKTTPPVLDSDGSFFLHSKLTVDKSRWQQGNVFS | |
| | CSVMHEALHNHYTQKSLSLSPGK | |
| 1A1-based | EVQLVQSGAEVKKPGASVKVSCKASGYTFTSYGISW | SEQ ID NO: 20 |
| GPC3×CD3 | VRQAPGQGLEWMGWISAYNGNTNYAQKLQGRVT | |
| HBiTE heavy | MTTDTSTSTAYMELRSLRSDDTAVYYCARAGTPTQI | |
| chain | LRYFDWLSQPFDYWGQGTLVTVSSGGGSSGGGGSG | |
| | GGGSEVQLVESGGGLVQPGGSLRLSCAASGFTFNTY | |
| | AMNWVRQAPGKGLEWVARIRSKYNNYATYYADSV | |
| | KGRFTISRDDSKNTLYLQMNSLRAEDTAVYYCARH | |
| | GNFGSSYVSYFAYWGQGTTVTVSSASTKGPSVFPLA | |
| | PSSKSTSGGTAALGCLVKDYFPEPVTVSWNSGALTS | |
| | GVHTFPAVLQSSGLYSLSSVVTVPSSSLGTQTYICNV | |
| | NHKPSNTKVDKKVEPKSCPPCPAPELLGGPSVFLFPP | |
| | KPKDTLMISRTPEVTCVVVDVSHEDPEVKFNWYVD | |
| | GVEVHNAKTKPREEQYNSTYRVVSVLTVLHQDWLN | |
| | GKEYKCKVSNKALPAPIEKTISKAKGQPREPQVYTLP | |
| | PSRDELTKNQVSLLCLVKGFYPSDIAVEWESNGQPE | |
| | NNYKTTPPVLDSDGSFFLHSKLTVDKSRWQQGNVFS | |
| | CSVMHEALHNHYTQKSLSLSPGK | |
| 6A4-based | DVVMTQSPSSLSASVGDRVTITCRASQSISSYLNWY | SEQ ID NO: 30 |
| GPC3×CD3 | QQKPGKAPKLLIYAASSLQSGVPSRFSGSGSGTDFTL | |
| HBiTE light | TISSLQPEDFATYYCQQSYSTPLTFGGGTKVEIKGSG | |
| chain | GGGSGGGGSEIVVTQSPATLSVSPGERATLSCRSSTG | |
| | AVTTSNYANWVQQKPGQAPRGLIGGANKRAPGVPA | |
| | RFSGSLSGDEATLTISSLQSEDFAVYYCALWYSNLW | |
| | VFGQGTKLEIKRTVAAPSVFIFPPSDEQLKSGTASVV | |
| | CLLNNFYPREAKVQWKVDNALQSGNSQESVTEQDS | |
| | KDSTYSLSSTLTLSKADYEKHKVYACEVTHQGLSSP | |
| | VTKSFNRGECPPCPAPELLGGPSVFLFPPKPKDTLMIS | |
| | RTPEVTCVVVDVSHEDPEVKFNWYVDGVEVHNAK | |
| | TKPREEQYNSTYRVVSVLTVLHQDWLNGKEYKCKV | |
| | SNKALPAPIEKTISKAKGQPREPQVYTLPPSRDELTK | |
| | NQVSLLCLVKGFYPSDIAVEWESNGQPENNYKTTPP | |
| | VLDSDGSFFLHSKLTVDKSRWQQGNVFSCSVMHEA | |
| | LHNHYTQKSLSLSPGK | |
| 6A4-based | EVQLVQSGAEVKKPGASVKVSCKASGYTFTSYAMH | SEQ ID NO: 31 |
| GPC3×CD3 | WVRQAPGQRLEWMGWINAGNGNTKYSQKFQGRVT | |
| HBiTE heavy | ITRDTSTSTAYMELRSLRSDDTAVYYCARDPSHWGQ | |
| chain | GTLVTVSSGGGSSGGGGSGGGGSEVQLVESGGGLV | |
| | QPGGSLRLSCAASGFTFNTYAMNWVRQAPGKGLEW | |
| | VARIRSKYNNYATYYADSVKGRFTISRDDSKNTLYL | |
| | QMNSLRAEDTAVYYCARHGNFGSSYVSYFAYWGQ | |
| | GTTVTVSSASTKGPSVFPLAPSSKSTSGGTAALGCLV | |
| | KDYFPEPVTVSWNSGALTSGVHTFPAVLQSSGLYSL | |
| | SSVVTVPSSSLGTQTYICNVNHKPSNTKVDKKVEPK | |
| | SCPPCPAPELLGGPSVFLFPPKPKDTLMISRTPEVTCV | |
| | VVDVSHEDPEVKFNWYVDGVEVHNAKTKPREEQY | |
| | NSTYRVVSVLTVLHQDWLNGKEYKCKVSNKALPAP | |
| | IEKTISKAKGQPREPQVYTLPPSRDELTKNQVSLLCL | |
| | VKGFYPSDIAVEWESNGQPENNYKTTPPVLDSDGSF | |
| | FLHSKLTVDKSRWQQGNVFSCSVMHEALHNHYTQK | |
| | SLSLSPGK | |

### Example 4. Binding Affinity of the Anti-GPC3 Monoclonal Antibodies to GPC3

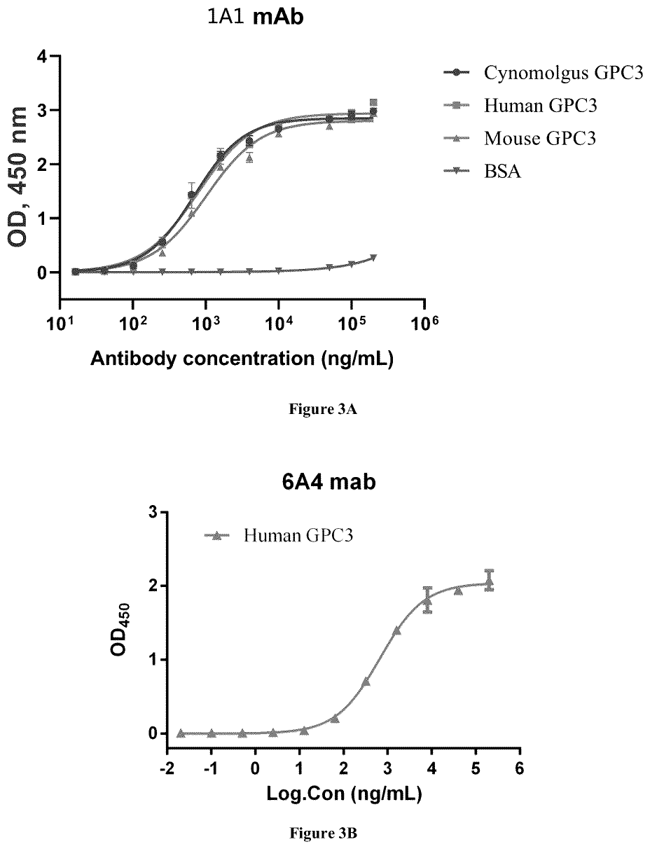

ELISA was performed according to standard protocols, to determine binding affinity of anti-GPC3 1A1 mAb to recombinant GPC3 from human, cynomolgus, and mouse and anti-GPC3 6A4 mAb to recombinant human GPC3. Briefly, recombinant GPC3 (AcroBiosystems) was coated on Corning EIA/RIA high-binding 96-well plates (Corning Inc.) at 50 ng per well overnight at 4° C. and blocked with 3% nonfat milk in PBS (pH7.4). Fivefold serially diluted biotinylated antibodies were added and incubated at room temperature for 2 h. The plates were washed with PBS containing 0.05% Tween 20. Bound antibodies were detected by HRP-conjugated streptavidin (Sino Biological). The assay was developed at room temperature with TMB substrate (Solarbio) and monitored at 450 nm with a microplate reader. The half-maximal binding (EC₅₀) was calculated by fitting the data to the Langmuir adsorption isotherm. The results are shown in FIG. 3A-3B.

As can be seen from FIG. 3A, the 1A1 mAb can bind to recombinant GPC3 from all three species with similar affinity. The EC₅₀s of 1A1 mAb binding to human, cynomolgus, and mouse GPC3 are 0.6 nM, 0.58 nM, and 1.12 nM, respectively, suggesting that the 1A1 mAb has high binding affinity to GPC3 proteins from different species. As can be seen from FIG. 3B, the 6A4 mAb binds to recombinant GPC3 with EC₅₀ of 4.5 nM.

### Example 5. Binding of the Anti-GPC3 Monoclonal Antibodies to Cell Surface-Associated GPC3 in Various Cancer Cell Lines

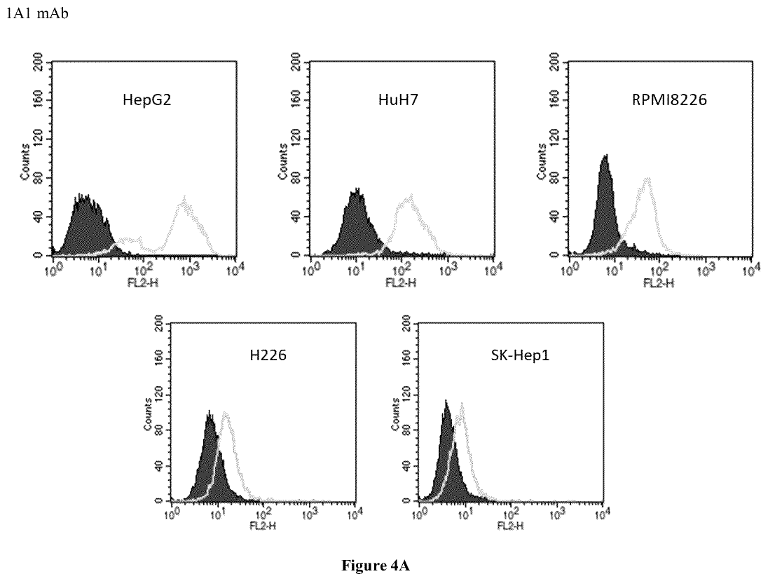

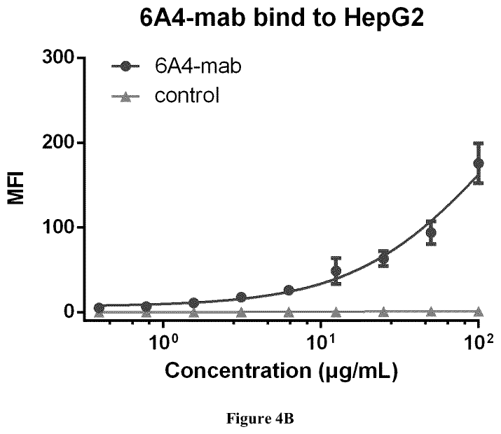

To measure binding ability of the anti-GPC3 1A1 mAb and 6A4 mAb to cell surface-associated GPC3, flow cytometry was carried out with multiple cancer cell lines including HepG2, HuH7, RPMI8226, H226, and SK-HEP-1. For 1A1 mAb, about 5×10⁵ cells of each of the cell lines were incubated with the antibody (10 μg/ml) on ice for 1 h. The cells were washed once with PBS containing 0.1% bovine serum albumin (PBSA) and resuspended in 100 μl PBSA. Then 1 μl anti-human IgG (Fc-specific)-FITC conjugate (Sigma) was added and incubated for 30 min. The cells were washed once with PBSA and then used for flow cytometry analysis. The results are shown in FIG. 4A.

For 6A4 mAb, HepG2 cells were digested with trypsin, centrifuged, and resuspend in 0.5% PBSA to a density of 5×10⁶ cells/mL. 90 μL of cell suspension was added to each EP tube. The anti-GPC3 6A4 mAb was prepared into a concentration of 2 mg/mL, and then diluted 2-fold serially to obtain working solutions. An IgG isotype antibody was used as negative control. 10 μL of various working solutions was added to each EP tube as above, mixed, and incubated at 4° C. for 60 min. After incubation, all EP tubes were centrifuged at 400 g for 5 min, washed twice with 0.5% PBSA. Then, the cells were resuspend in 100 μL of 0.5% PBSA, added with 2 μL of anti-human IgG (γ-chain specific)-R-phycoerythrin antibody, and incubated at 4° C. for 30 min in the dark. After incubation with the secondary antibody, the cells were centrifuged and washed twice, and resuspend in 400 μL of 0.5% PBSA for flow cytometry. The results are shown in FIG. 4B.

As can be seen from FIG. 4A, 1A1 mAb binds well to Hep-G2, HuH7 and RPMI8226, while shows moderate binding to H226 and SK-HEP1. As can be seen from FIG. 4B, 6A4 mAb can bind to the GPC3 positive tumor cell line HepG2. This suggests that 1A1 mAb and 6A4 mAb have ability of binding to GPC3 positive tumor cell lines.

### Example 6. Binding Affinity of the Bispecific Antibodies Targeting GPC3 and CD3 to GPC3 and CD3

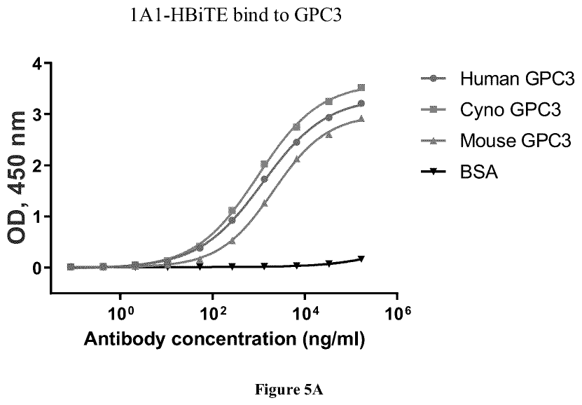

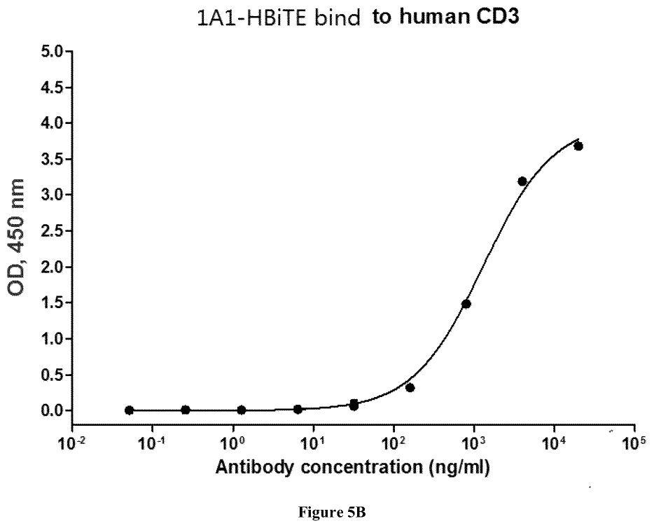

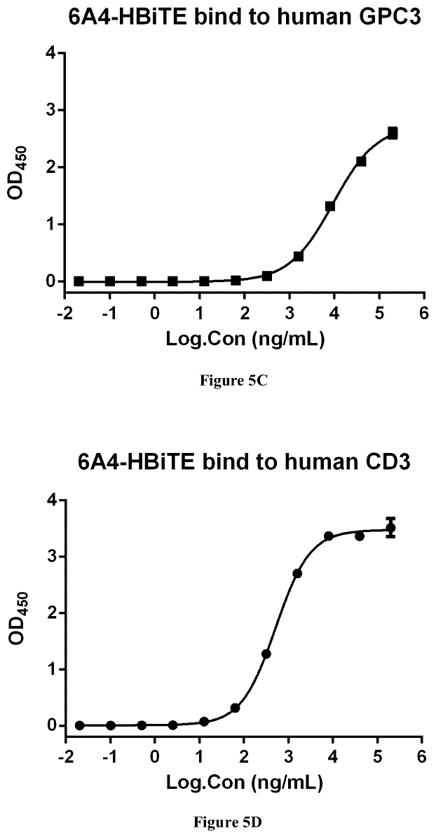

To determine binding affinity of the bispecific antibodies 1A1-based GPC3×CD3 HBiTE and 6A4-based GPC3×CD3 HBiTE to both GPC3 and CD3, ELISA was performed as described in Example 4, with the coating proteins of human, cynomolgus or mouse GPC3, or human CD3. The results are shown in FIGS. 5A-5D.

The results indicate that the 1A1-based GPC3×CD3 HBiTE binds to human, cynomolgus, and mouse GPC3 with EC₅₀ of 48.56 nM, 41.21 nM, and 69.84 nM, respectively (FIG. 5A), and binds to human CD3 with EC₅₀ of 10.8 nM (FIG. 5B). The 6A4-based GPC3×CD3 HBITE binds to human GPC3 with EC₅₀ of 75.2 nM (FIG. 5C), and binds to human CD3 with EC₅₀ of 4.2 nM (FIG. 5D).

These results suggested that the bispecific antibodies can bind to both GPC3 and CD3 proteins, with affinity suitable for been used as BiTE to trigger tumor cell killing by T cells.

### Example 7. Binding of the Bispecific Antibodies Targeting GPC3 and CD3 to Cancer Cell Lines

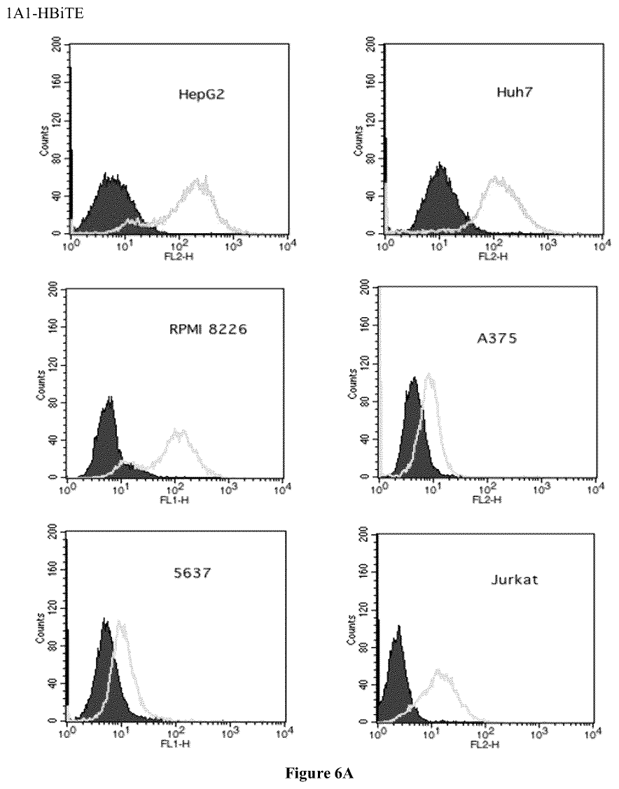

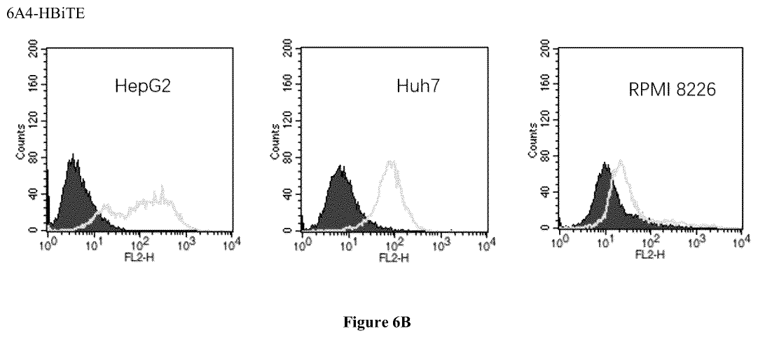

To determine binding affinity of the bispecific antibodies 1A1-based GPC3×CD3 HBITE and 6A4-based GPC3×CD3 HBiTE to cancer cell lines, flow cytometry was carried out with multiple GPC3 expressing cancer cell lines including Hep-G2, HuH7, RPMI8226, A375, 5637, and CD3 positive Jurkat cell line. The procedures were similar to those described in Example 5. The results were shown in FIG. 6A-6B.

The results indicate that the 1A1-based GPC3×CD3 HBiTE binds well to HepG2, HuH7, RPMI8226 and CD3 expressing Jurkat cells, and has moderate binding to A375 and 5637 (FIG. 6A); while the 6A4-based GPC3×CD3 HBiTE binds well to HepG2 and HuH7 and has moderate binding to RPMI8226 (FIG. 6B). This suggests that 1A1-based GPC3×CD3 HBiTE and 6A4-based GPC3×CD3 HBiTE can bind to both cancer cells expressing GPC3 and cells expressing CD3.

### Example 8. Bispecific Antibodies Mediated Killing of Human Cancer Cell Lines In Vitro

Bispecific T cell engager can simultaneously bind to a tumor antigen and a T cell antigen (e.g., CD3 molecular on T cell surface) causing aggregation and activation of T cells, eventually leading to the killing of tumor cells. To evaluate killing efficiency of the bispecific antibody 1A1-based GPC3×CD3 HBiTE, four GPC3 expressing cell lines HepG-2, HuH7, RPMI-8226 and LS174T-GPC3 were used as target cells.

For human liver cancer cell lines HepG2 and Huh-7, killing assay was performed by monitoring electrical impedance of cells by using a Maestro ZHT platform (Axion BioSystems). 100 μL of cell suspension (2000 cells/well, suspended in RPMI 1640 complete medium) was seeded in triplicate into a 384-well plate. The plate was pre-incubated for 24 hours in Maestro ZHT platform. Meanwhile, frozen stocked PBMCs were revived and resuspended in RPMI 1640 complete medium. The target cells were incubated for 24 hours at 37° C. and 5% CO₂ in the incubator. At the second day, 50 μL of culture supernatant was discarded and 10⁴ PBMCs in 25 μL RPMI 1640 complete medium (target:effector ratio=1:5) were added into each well. Then, 25 μl antibody (5-fold serially diluted from 20 μg/mL) was added into each well (the highest final concentration was 5 μg/mL), accordingly. 48 hours after treatment, an endpoint was set and the data of cell electrical impedance (Z) was exported. Cell growth inhibition was calculated by the following equation:

$$cell growth inhibition rate ( \% ) = ( {Z}_{control} - {Z}_{exp} ) / {Z}_{control} \times 100 \% ;$$

in which, Z_{control} represents cell electrical impedance of control group, and Zₑₓₚ represents cell electrical impedance of experimental group.

For human myeloma cell line RPMI8226, both LDH and CCK8 assay were performed to test killing efficiency according to manufacturer's instructions. 100 μL of cell suspension (3×10⁴ cells/well, suspended in RPMI 1640 complete medium) was seeded in duplicate into a 96-well plate. Meanwhile, 1.5×10⁵ PBMCs in 50 μL RPMI 1640 complete medium (target:effector ratio=1:5) were added. Then, 50 μL of 5-fold serially diluted antibody solutions (from 0.8 μg/ml) were added into each well (the highest final concentration was 0.2 μg/mL), accordingly. 48 hours after treatment, the 96-well plate was centrifuged and 100 μL of the culture supernatant was collected to detect the optical density (OD) value at 490 nm (OD490) according to the instruction of Cytotoxicity LDH Assay Kit-WST. Cell growth inhibition was calculated by the following equation:

$$cell growth inhibition rate ( \% ) = ( {OD}_{exp} - {OD}_{low control} ) / ( {OD}_{high control} - {OD}_{low control} ) \times 100 \% ;$$

in which, ODₑₓₚ represents the OD490 value of experimental group, OD_{low control} represents the OD490 value of a control group of live cells, and OD_{high control} represents the OD490 value of a control group that live cells were all killed by lysis buffer.

Concurrently, the remaining cell culture in each well was supplemented with 100 μL RPMI 1640 complete medium containing 20% CCK-8 (the final concentration was 10% CCK-8) and incubated in a CO₂ incubator for 60 minutes. Optical density (OD) values at 490 nm were read by a microplate reader. Cell growth inhibition was calculated by the following equation:

$$cell growth inhibition rate ( \% ) = ( {OD}_{control} - {OD}_{exp} ) / {OD}_{control} \times 100 \% ;$$

in which, OD_{control} represents the OD490 value of control group, and ODₑₓₚ represents the OD490 value of experimental group.

For human colon adenocarcinoma cell line LS174T-GPC3, 100 μL of cell suspension (3×10⁴ cells/well, suspended in RPMI 1640 complete medium) was seeded in duplicate into a 96-well plate. Meanwhile, 1.5×10⁵ PBMCs in 50 μL RPMI 1640 complete medium (target:effector ratio=1:5) were added. Then, 50 μL of 5-fold serially diluted antibody solutions (from 0.8 μg/ml) were added into each well (the highest final concentration was 0.2 μg/mL), accordingly. After 48 h, each well was supplemented with 100 μL RPMI 1640 complete medium containing 20% CCK-8 (the final concentration was 10% CCK-8) and incubated in a CO₂ incubator for 60 minutes. Optical density (OD) values at 490 nm were read by a microplate reader. Cell growth inhibition was calculated by the following equation:

$$cell growth inhibition rate ( \% ) = ( ODcontrol - ODexp ) / ODcontrol \times 100 \% ;$$

in which, OD_{control} represents the OD490 value of control group, and ODₑₓₚ represents the OD490 value of experimental group.

To evaluate killing efficiency of the bispecific antibody 6A4-based GPC3×CD3 HBiTE, the GPC3 expressing cell line HuH7 was used as target cells. 100 μL of cell suspension (1.2×10⁴ cells/well, suspended in RPMI 1640 complete medium) was seeded in duplicate into a 96-well plate. The plate was pre-incubated for 24 hours at 37° C. and 5% CO₂ in an incubator. Meanwhile, PBMCs were revived and resuspended in RPMI 1640 complete medium. The cells were incubated for 24 hours in the incubator. At the second day, 1.5×10⁵ PBMCs in 50 μL RPMI 1640 complete medium (target:effector ratio=1:12.5) were added into the 96-well plate. Then, 50 μl antibodies (5-fold serially diluted from 4 μg/mL) were added into each well (the highest final concentration was 1 μg/mL). 48 hours after treatment, cells in each well were collected and incubated with the first antibody (GPC3-Mab, 20 μg/mL) for 1 hour at 4° C. Next, the cells were washed and incubated with the secondary antibody (Anti-Human IgG (γ-chain specific)-R-Phycoerythrin antibody produced in goat) for another 30 minutes at 4° C. Finally, the cells were transferred to BD Trucount™ Tubes, acquired and analyzed by BD FACS Calibur and BD CellQuest Pro, respectively.

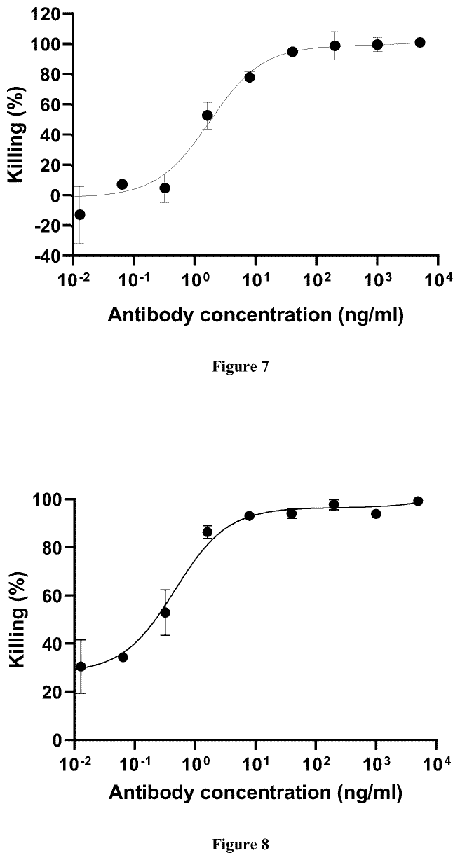

For human liver cancer cell lines HepG2 and Huh7, the results show that nearly 100% tumor cells were killed in the presence of 1A1-based GPC3×CD3 HBITE and PBMC. The EC₅₀ of HepG2 killing by 1A1-based GPC3×CD3 HBiTE was 1.762 ng/ml (FIG. 7), and the EC₅₀ of HuH7 killing by 1A1-based GPC3×CD3 HBiTE was 0.491 ng/ml (FIG. 8). These results suggest that the 1A1-based GPC3×CD3 HBiTE has potent killing efficiency against both Hep-G2 and HuH7 cells.

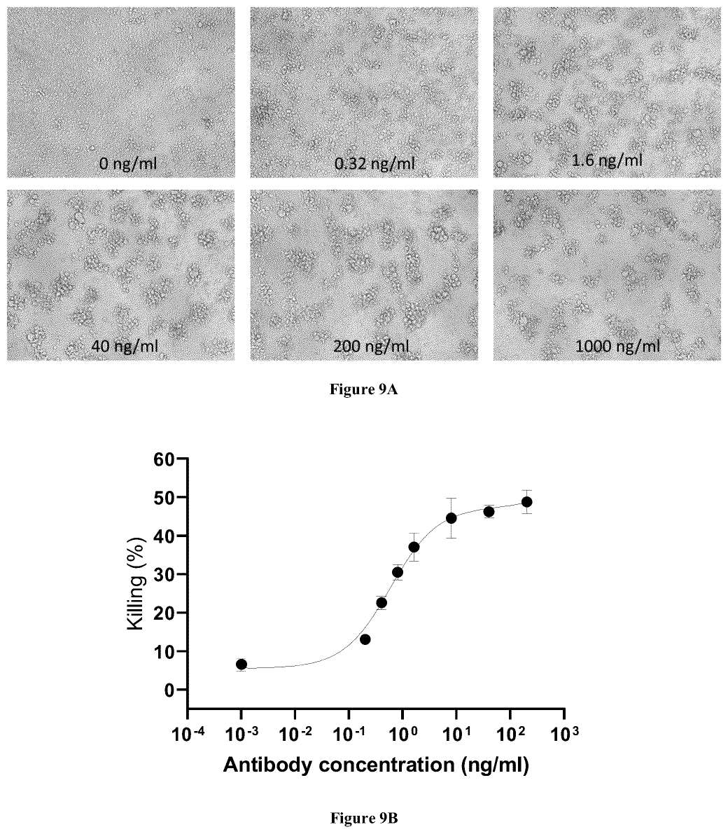

For human myeloma cell line RPMI8226, the two assays yielded consistent result: around 50% tumor cells were killed by 1A1-based GPC3×CD3 HBiTE in the presence of PBMC. FIG. 9A shows the formation of tumor cell clusters after the 1A1-based GPC3×CD3 HBITE was added. The EC₅₀ of RPMI8226 killing by 1A1-based GPC3×CD3 HBiTE was 0.589 ng/ml (FIG. 9B).

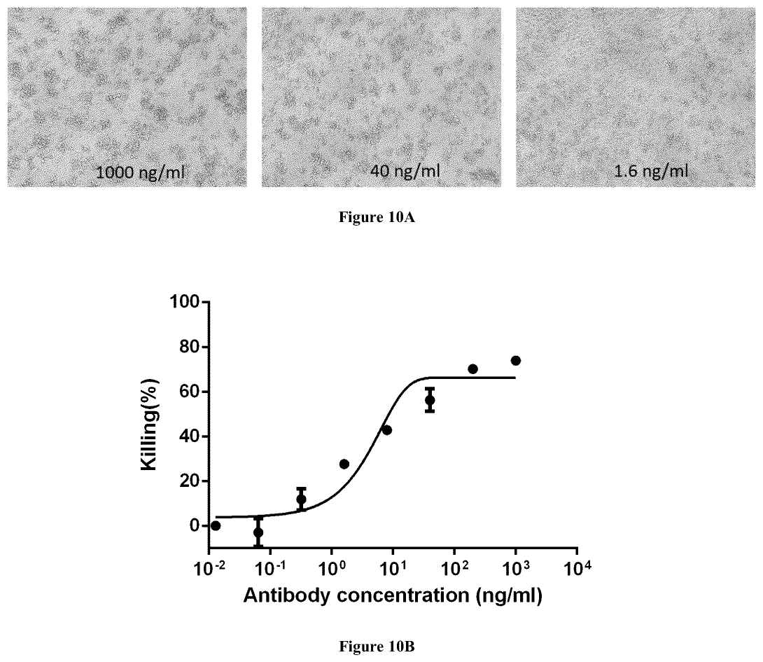

The killing of 1A1-based GPC3×CD3 HBITE against LS174T-GPC3 cells was shown in FIG. 10A-10B. FIG. 10A shows the formation of tumor cell clusters after the 1A1-based GPC3×CD3 HBiTE was added. FIG. 10B shows that nearly 70% tumor cells were killed in the presence of 1A1-based GPC3×CD3 HBiTE and PBMC. The EC₅₀ of LS174T-GPC3 killing by 1A1-based GPC3×CD3 HBiTE was 1.25 ng/ml (FIG. 10B). This suggests that the 1A1-based GPC3×CD3 HBiTE has potent killing efficiency against LS174T-GPC3 cells.

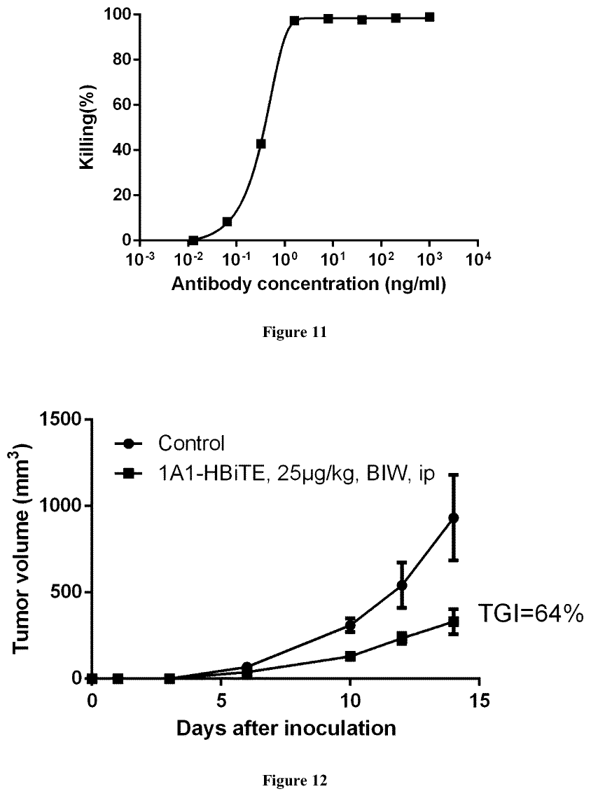

The killing of 6A4-based GPC3×CD3 HBiTE against HuH7 cells was shown in FIG. 11. The results show that nearly 100% tumor cells were killed in the presence of 6A4-based GPC3×CD3 HBiTE and PBMC. The EC₅₀ of HuH7 killing by 6A4-based GPC3×CD3 HBiTE was 1.14 ng/ml. This suggests that the 6A4-based GPC3×CD3 HBiTE has potent killing efficiency against HuH7 cells.

Taken together, the results have demonstrated that the 1A1-based GPC3×CD3 HBiTE and 6A4-based GPC3×CD3 HBiTE possess potent killing capability against multiple cancer cell lines including human liver cancer cell lines, human myeloma cell line and human colon adenocarcinoma cell line, suggesting good potential for treating various cancers expressing GPC3.

### Example 9. Bispecific Antibodies Mediated Killing of Human Cancer Cell Lines In Vivo

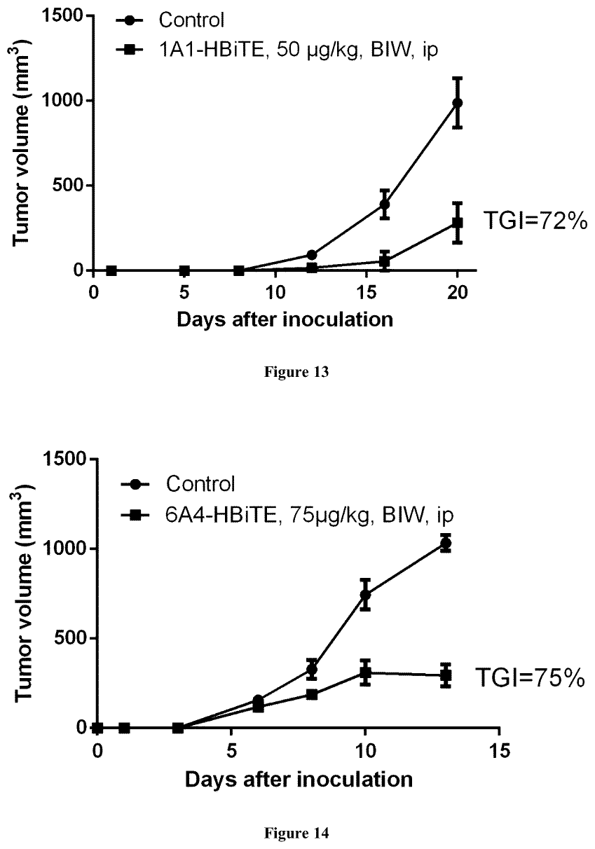

To evaluate killing efficacy of 1A1-based GPC3×CD3 HBiTE, in vivo anti-tumor experiment was performed in a humanized B-NDG model (5-7 weeks old, male). Briefly, 1×10⁷ PBMCs were injected intravenously (i.v.) into B-NDG mice to create Hu-PBL models (Day-7). Starting on the day 0, 1×10⁶ LS174T-GPC3 (or 3×10⁶ Huh-7) tumor cells were injected subcutaneously on the right flank of the mice. Meanwhile, 1A1-based GPC3×CD3 HBITE (25 μg/kg for LS174T-GPC3; 50 μg/kg for Huh-7) or vehicle control was injected intraperitoneally to the mice twice a week. After treatment, tumor size was continuously measured for 2-3 weeks. The results were shown in FIGS. 12-13.

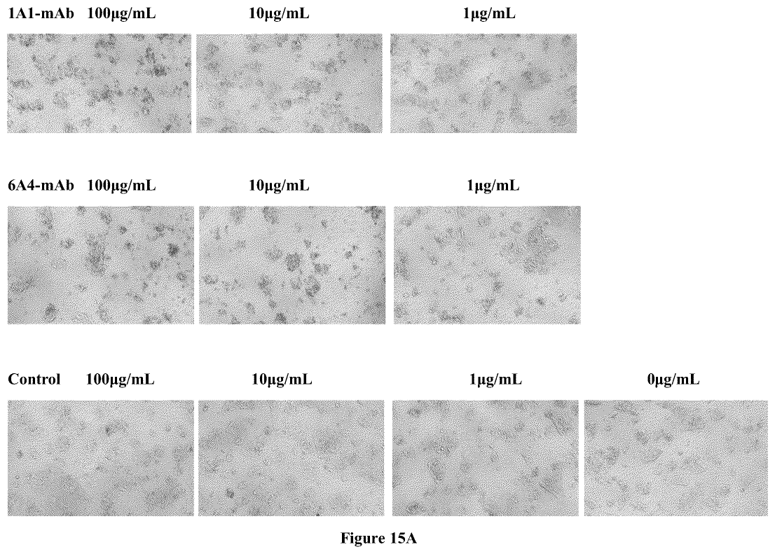

To evaluate killing efficacy of 6A4-based GPC3×CD3 HBiTE, in vivo anti-tumor experiment was performed in a humanized PBMC/B-NDG model. Briefly, 1×10⁶ LS174T-GPC3 tumor cells were mixed with 1×10⁶ human PBMCs and Corning Matrigel, then injected subcutaneously on the right flank of Hu-PBL mice. Starting on the second day (treatment day 1), 6A4-based GPC3×CD3 HBITE (75 μg/kg) or vehicle control was injected intraperitoneally to the mice three times a week. After treatment, tumor size was continuously measured for 2 weeks. The results were shown in FIG. 14.

As can be seen from FIGS. 12-13, administration of 1A1-based GPC3×CD3 HBiTE results in significantly decreased tumor volume in both LS174T-GPC3 and Huh-7 cells, compared with the control group. As can be seen from FIG. 14, administration of 6A4-based GPC3×CD3 HBiTE results in significantly decreased tumor volume in LS174T-GPC3 cells, compared with the control group. These results indicate that the 1A1-based GPC3×CD3 HBiTE and 6A4-based GPC3×CD3 HBiTE possess potent killing capability against multiple cancer cell lines expressing GPC3 and can be used for treating various cancers expressing GPC3.

### Example 10. Anti-GPC3 Monoclonal Antibodies Mediated ADCC Killing Against Human Cancer Cell Lines

Frozen NK cells were revived and cultured in RPMI1640 complete medium containing 20% FBS, 1% penicillin/streptomycin and 50 IU IL-2 overnight at 37° C. and 5% CO₂. HepG2 cells were used as target cells and diluted to a concentration of 2.5×10⁵ cells/mL with the complete medium, and added to a 96-well plate at 100 μL/well and cultured overnight at 37° C. Anti-GPC3 monoclonal antibodies 1A1 mAb and 6A4 mAb were prepared to concentrations of 400 μg/mL, 40 μg/mL and 4 μg/mL, respectively, with RPMI1640 medium, and an IgG isotype antibody was used as negative control. The prepared antibody solutions were added to the 96-well plate containing target cells at 50 μL/well. NK cells were collected by centrifugation and diluted to 1×10⁶ cells/mL with the complete medium. 50 μL of NK cells were added to the 96-well plate. The final concentrations of the antibodies were 100 μg/mL, 10 μg/mL and 1 μg/mL, respectively. All culture plates were incubated at 37° C. for 72 h. Then, the original medium was removed and replaced with fresh medium containing 10% CCK-8 at 100 μL/well. The plates were incubated at 37° C. for about 30 min, and measured for OD values using a microplate reader at 450 nm (reference wavelength was 630 nm).

Killing efficiency was calculated by the following equation:

$$Cytotoxicity \% = ( {OD}_{Tumor + NK + 0 μ g / mL mab} - {OD}_{Tumor + NK + x μ g / mL mab} ) / {OD}_{Tumor + NK + 0 μ g / mL mab} \times 100 \% ;$$

in which x represents 1, 10 or 100.

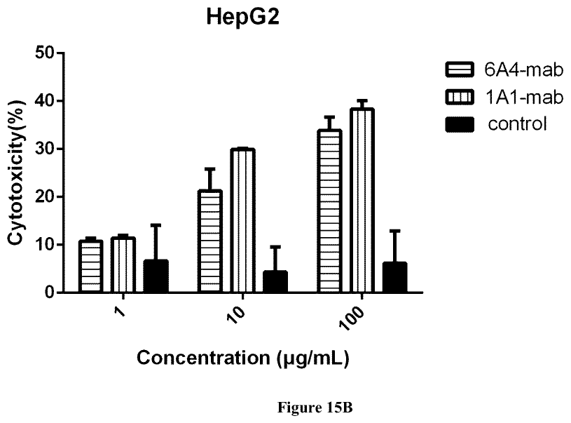

The ADCC killing of 1A1 mAb and 6A4 mAb against HepG2 cells was shown in FIGS. 15A-15B. The results indicate that 1A1 mAb and 6A4 mAb mediate significantly increased ADCC killing against HepG2 cells, compared with the control group, and the killing efficiency is dose-dependent. This suggests that the 1A1 mAb and 6A4 mAb have potent killing efficiency against cancer cell lines expressing GPC3.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. An antibody specifically binding to GPC3, or an antigen binding fragment thereof, comprising a light chain variable region (VL) and a heavy chain variable region (VH), wherein
(i) the VL comprises LCDRs 1-3 having the amino acid sequences as set forth in SEQ ID NOs: 1-3 respectively, and the VH comprises HCDRs 1-3 having the amino acid sequences as set forth in SEQ ID NOs: 6-8 respectively; or
(ii) the VL comprises LCDRs 1-3 having the amino acid sequences as set forth in SEQ ID NOs: 23-25 respectively, and the VH comprises HCDRs 1-3 having the amino acid sequences as set forth in SEQ ID NOs: 60-62 respectively.

2. The antibody or the antigen binding fragment thereof according to claim 1, wherein
(i) the VL comprises an amino acid sequence as set forth in SEQ ID NO: 4 or an amino acid sequence having at least 80%, at least 85%, at least 90%, at least 95%, at least 98%, or at least 99%, sequence identity to SEQ ID NO: 4, and the VH comprises an amino acid sequence as set forth in SEQ ID NO: 9 or an amino acid sequence having at least 80%, at least 85%, at least 90%, at least 95%, at least 98%, or at least 99%, sequence identity to SEQ ID NO: 9; or
(ii) the VL comprises an amino acid sequence as set forth in SEQ ID NO: 26 or an amino acid sequence having at least 80%, at least 85%, at least 90%, at least 95%, at least 98%, or at least 99% sequence identity to SEQ ID NO: 26, and the VH comprises an amino acid sequence as set forth in SEQ ID NO: 28 or an amino acid sequence having at least 80%, at least 85%, at least 90%, at least 95%, at least 98%, or at least 99% sequence identity to SEQ ID NO: 28.

3. (canceled)

4. The antibody or the antigen binding fragment thereof according to claim 1, wherein the antibody is of an isotype selected from the group consisting of IgG, IgA, IgM, IgE and IgD.

5. The antibody or the antigen binding fragment thereof according to claim 1, wherein the antibody is of a subtype selected from the group consisting of IgG1, IgG2, IgG3, and IgG4.

6. The antibody or the antigen binding fragment thereof according to claim 1, wherein the antigen binding fragment is selected from the group consisting of Fab, Fab′, F(ab′)₂, Fv, scFv, and ds-scFv.

7. The antibody or the antigen binding fragment thereof according to claim 1, wherein the antibody is a monoclonal antibody.

8. The antibody or the antigen binding fragment thereof according to claim 7, wherein the antibody comprises
(i) a light chain comprising an amino acid sequence as set forth in SEQ ID NO: 5 or an amino acid sequence having at least 80%, at least 85%, at least 90%, at least 95%, at least 98%, or at least 99% sequence identity to SEQ ID NO: 5 and a heavy chain comprising an amino acid sequence as set forth in SEQ ID NO: 10 or an amino acid sequence having at least 80%, at least 85%, at least 90%, at least 95%, at least 98%, or at least 99% sequence identity to SEQ ID NO: 10; or
(ii) a light chain comprising an amino acid sequence as set forth in SEQ ID NO: 27 or an amino acid sequence having at least 80%, at least 85%, at least 90%, at least 95%, at least 98%, or at least 99% sequence identity to SEQ ID NO: 27 and a heavy chain comprising an amino acid sequence as set forth in SEQ ID NO: 29 or an amino acid sequence having at least 80%, at least 85%, at least 90%, at least 95%, at least 98%, or at least 99% sequence identity to SEQ ID NO: 29.

9. (canceled)

10. The antibody or the antigen binding fragment thereof according to claim 9, wherein the antibody is a bispecific antibody which further comprises a second antigen binding region binding to a second antigen.

11. The antibody or the antigen binding fragment thereof of according to claim 10, wherein the second antigen is a tumor associated antigen, an immune cell antigen, or a T-cell antigen.

12. (canceled)

13. The antibody or the antigen binding fragment thereof according to claim 12, wherein the T-cell antigen is selected from the group consisting of T cell receptor (TCR), CD3, CD4, CD8, CD16, CD25, CD28, CD44, CD62L, CD69, ICOS, 41-BB (CD137), and NKG2D.

14. The antibody or the antigen binding fragment thereof according to claim 10, wherein the second antigen is CD3, and the second antigen binding region comprises a VL and a VH, wherein the VL comprises LCDRs 1-3 having the amino acid sequences as set forth in SEQ ID NOs: 11-13 respectively, and the VH comprises HCDRs 1-3 having the amino acid sequences as set forth in SEQ ID NOs: 16-18 respectively.

15. The antibody or the antigen binding fragment thereof according to claim 14, wherein the second antigen binding region comprises a VL comprising an amino acid sequence as set forth in SEQ ID NO: 14 or an amino acid sequence having at least 80%, at least 85%, at least 90%, at least 95%, at least 98%, or at least 99% sequence identity to SEQ ID NO: 14 and a VH comprising an amino acid sequence as set forth in SEQ ID NO: 19 or an amino acid sequence having at least 80%, at least 85%, at least 90%, at least 95%, at least 98%, or at least 99% sequence identity to SEQ ID NO: 19.

16. (canceled)

17. The antibody or the antigen binding fragment thereof according to claim 14, wherein the VL of the second antigen binding region is linked to the C-terminal of the VL of the antibody specifically binding to GPC3, optionally via a first linker, and the VH of the second antigen binding region is linked to the C-terminal of the VH of the antibody specifically binding to GPC3, optionally via a second linker, wherein the first linker and the second linker are the same or different.

18. The antibody or the antigen binding fragment thereof according to claim 17, wherein the first linker comprises an amino acid sequence as set forth in SEQ ID NO: 21 or SEQ ID NO: 32, and the second linker comprises an amino acid sequence as set forth in SEQ ID NO: 22.

19. The antibody or the antigen binding fragment thereof according to claim 14, wherein the bispecific antibody comprises
(i) a light chain comprising an amino acid sequence as set forth in SEQ ID NO: 15 or an amino acid sequence having at least 80%, at least 85%, at least 90%, at least 95%, at least 98%, or at least 99% sequence identity to SEQ ID NO: 15 and a heavy chain comprising an amino acid sequence as set forth in SEQ ID NO: 20 or an amino acid sequence having at least 80%, at least 85%, at least 90%, at least 95%, at least 98%, or at least 99% sequence identity to SEQ ID NO: 20; or
(ii) a light chain comprising an amino acid sequence as set forth in SEQ ID NO: 30 or an amino acid sequence having at least 80%, at least 85%, at least 90%, at least 95%, at least 98%, or at least 99% sequence identity to SEQ ID NO: 30 and a heavy chain comprising an amino acid sequence as set forth in SEQ ID NO: 20 or an amino acid sequence having at least 80%, at least 85%, at least 90%, at least 95%, at least 98%, or at least 99% sequence identity to SEQ ID NO: 31.

20-27. (canceled)

28. A nucleic acid comprising a nucleotide sequence encoding the antibody or the antigen binding fragment thereof according to claim 1.

29-30. (canceled)

31. A pharmaceutical composition comprising (i) the antibody or the antigen binding fragment thereof according to claim 1; and (ii) a pharmaceutically acceptable carrier or excipient.

32-34. (canceled)

35. A conjugate, comprising the antibody or the antigen binding fragment thereof according to claim 1 and a chemical moiety conjugated thereto.

36. (canceled)

37. A method of treating a cancer in a subject, comprising administering to the subject an effective amount of the antibody or the antigen binding fragment thereof according to claim 1, wherein the cancer is a GPC3 positive cancer.

38. (canceled)

39. The method according to claim 37, wherein the cancer is selected from the group consisting of liver cancer, colon cancer, pancreatic cancer, lung cancer, bladder cancer, melanoma and myeloma.

40-42. (canceled)
